(19) United States
(12) Patent Application Publication
(10) Pub. No.: US 2010/0105102 A1
(43) Pub. Date: Apr. 29, 2010

(54) DEVICE, METHOD AND APPARATUS FOR ANALYZING SKIN AND HAIR

(75) Inventors: Robert Eugene Hanes, Austin, TX (US); J. Brian Windsor, Austin, TX (US); Damon V. Borich, Austin, TX (US); Jason Neeser, Austin, TX (US); Michael Rasoulian, Austin, TX (US); P. Rogelio Escamilla, Kyle, TX (US)

Correspondence Address: CHALKER FLORES, LLP, 2711 LBJ FRWY, Suite 1036, DALLAS, TX, 75234, US

(73) Assignee: Reveal Sciences, LLC, Austin, TX (US)

(21) Appl. No.: 12/604,162

(22) Filed: Oct. 22, 2009

Related U.S. Application Data
(60) Provisional application No. 61/107,633, filed on Oct. 22, 2008.
(60) Provisional application No. 61/107,985, filed on Oct. 23, 2008.

(51) Int. Cl. C12Q 1/02; A61B 10/02; C12M 1/26; C12M 1/34

(52) U.S. Cl. 435/29; 600/562; 435/309.1; 435/287.1

(57) ABSTRACT

The present invention includes compositions, methods, and systems for the analysis of skin and hair conditions. The system includes a method and apparatus for analyzing skin and hair samples by taking a sample, identifying desired components of the sample, obtaining an image electronically, storing the image, and analyzing the image using analysis software.

### CROSS-REFERENCE TO RELATED APPLICATIONS

This application claims priority to U.S. Provisional Application Ser. Nos. 61/107,633, filed Oct. 22, 2008; and 61/107,985, filed Oct. 23, 2008, the entire contents of which are incorporated herein by reference.

### TECHNICAL FIELD OF THE INVENTION

The present invention relates in general to the field of surface analysis, and more particularly, to a novel system, method and apparatus for identifying biological markers to analyze skin and hair.

### STATEMENT OF FEDERALLY FUNDED RESEARCH

None.

### BACKGROUND OF THE INVENTION

Without limiting the scope of the invention, this invention relates generally to the field of skin and hair analysis, and, more particularly, to the development of a method and apparatus for identifying biological markers for the analysis of skin and hair conditions. Skin and hair analysis is important and greatly desired. Factors such as aging, physical condition, environmental stress, seasonal changes, hormonal fluctuation, biochemical irregularities, and other variables contribute to skin conditions and skin problems. Skin analysis tools assist professionals or individual customers in determining skin type and potential factors contributing to skin conditions. The resulting information allows the customer to choose the most appropriate cosmetic or personal care products in order to maintain or improve skin conditions.

Four general types of methods for skin analysis are currently available. First, questionnaires are used to make a determination of a patient's perceptions of their skin type, condition and needs. The answers to these questionnaires are processed to match a patient's apparent needs with certain pre-determined and pre-packaged skin care products. Second, tape stripping products are used to take a standard visual image of the skin that is compared to a standard visual image of various skin types to determine skin type. Based on the visual matching of the skin type one of products is selected for use. Third, scope or sensors are used to magnify the skin or make determinations of skin water contact, “oiliness” and elasticity. Again, based on these few physical parameters skin care products are selected. Fourth, 3-D imaging of the skin surface is used to determine skin melanin content, subcutaneous blood flow (by detecting hemoglobin), pore size, skin tone, bacterial content and even skin damage. Again, based on the analysis of these physical parameters, skin care products are selected.

One such system is taught in U.S. Pat. No. 7,211,043, issued to Pruche, et al. for a method, system and device for evaluating skin type. Briefly, the patent is direct to a method, device and system for determining skin type. The method includes a step of applying at least one drop of substance onto a zone of the skin or on a collector member previously in contact with the zone of the skin. The substance can modify at least one physicochemical property of the surface of the zone or of the collector member exposed to the substance. After the drop has disappeared or been removed, the extent of the surface is evaluated and the skin type is determined as a function of this evaluation.

While several methods for analyzing skin are currently available, including imaging and analysis of physical factors, none have used biological markers for determining variables applicable to determining skin and hair health and condition.

### SUMMARY OF THE INVENTION

The needs of the invention set forth above as well as further and other needs and advantages of the present invention are achieved by the embodiments of the invention described herein below. Briefly, the present invention includes compositions, methods and systems for the determination of skin surface biochemical content and characteristics that are not attainable using technology currently available. The system must conform to current technology and methods of use to maximize user compliance. It has been found, remarkably, that a relatively small sample of biochemical skin surface markers serve as surrogates for overall skin condition and treatment options. While hundreds of biochemical parameters could be obtained and explored, the present invention provides both in-depth knowledge, but makes it possible to minimize the parameters that provide maximal results. Furthermore, it was also found that the method and system of the present invention also provide a comprehensive understanding of hair condition.

More particularly, the present invention includes compositions, systems and methods for analyzing skin and hair sample biochemistry.

The present invention includes a surface sampling device comprising: a backing and at least two adhesive regions on the backing, wherein the adhesive in the adhesive regions is selected to capture a surface sample, wherein one or more agents are disposed in the adhesive region capable of interacting with the surface sample to aid in measuring one or more components of the sample. In one aspect, the sample comprises skin and/or or comprises one or more chemical species. In another aspect, the backing comprises a disposable card comprised from cardboard or vinyl sized for a cartridge. In another aspect, the adhesive is selected to remove skin cell from the stratum corneum. In one aspect, the device further comprises one or more membranes selected from nitrocellulose, UVPE, PVDF, hydrophobic membranes known to those skilled in the art of immunosorbent assays. In another aspect, the adhesive surface and the backing surface are selected to maximize the imaging capabilities of an imaging device through minimizing, maximizing or mixing reflective, absorbance and transmittance properties. In another aspect, the device further comprises an optical barcode (unique id). In another aspect, the backing comprises a background with a random colored pattern for security, calibration and test validation interpretable by an algorithm processing digital signal from a camera. In another aspect, backing comprises a thermochromic background material responsive to heat. In another aspect, the backing comprises at least one of an agent that is responsive to pressure and changes color; a translucent material that allows a simultaneous measurement of transmitted and reflected light; that is electrochromic; that changes color due to wetting, or pH or other specific chemical reaction; that comprises a phosphorescent compound that provides spontaneous illumination when expose to light of specific wavelength; comprises a chemiluminescent activated by a skin metabolite that then transmits light through regions absence of skin; and that responds to mechanical deformation by releasing embedded capsules of an activating chemical, dye or buffer.

In another embodiment, the present invention is a sampling device with two or more adhesive regions that have varying adhesive qualities such that upon use the sampling device gathers different amounts of sample in distinct regions. In another aspect, the adhesive surface comprises at least one of a preloaded region with an analyte specific reagent, such as a synthetic receptor; releases a dye upon experiencing a change in pressure; comprises a chemical composition for indicating health conditions; or allows flow to a subsequent surface. In another aspect, the device comprises an MIP adhesive thin film sandwiched beneath a backing material and an adhesive for removing tissue.

In another embodiment, the present invention is an adhesive composition for removing proteins, removing oils or capturing enzymes comprising an adhesive for removing the stratum corneum in different thicknesses for chemical testing. In another aspect, the adhesive substance embedded with a functional chemical for enhancing the image of a collected sample. In another aspect, the adhesive comprises an optical dye that emits in the visible range. In another aspect, the adhesive comprises an optical dye that selectively interacts with protein, oxidized protein or lipids. In another aspect, the adhesive comprises natural adhesives synthetic adhesives, drying adhesives, contact adhesives thermoplastic adhesives, reactive adhesives, UV and light curing adhesives or pressure sensitive adhesives. In another aspect, the adhesive comprises chemical and physical properties that detector one or more chemical species in the skin, is optimized for optical imaging, illuminated from LED's in the IR, visible and UV wavelengths. In another aspect, the adhesive further comprises one or more indicating dyes, buffers and activators for indicating the presence of skin markers. In another aspect, the adhesive comprises dyes, buffers and activators for highlighting a group of skin properties such as moisture, dryness, irritation, wrinkles or sun damage. In another aspect, the adhesive comprises two or more adhesives selected are porous, hydrophilic, hydrophobic, double-sided and hydrogels. In another aspect, the adhesive captures topographical information from the surface to which it is attached. In another aspect, the adhesive comprises one or more agents that simultaneously monitors pH, moisture content, and oil. In another aspect, the adhesive further comprises an agent that disrupts cell membranes. In another aspect, the adhesive further comprises one or more reagents selected from: a buffer, a dye, an activator a synthetic receptor or linker.

In another embodiment, the present invention is an assay comprising and adhesive for sample collection that comprises a receptor and a dye whose optical properties change in the presence of oxidized protein. In another embodiment, the present invention is a module comprising an adhesive for obtaining a sample device, comprising: a tape strip collected sample washed from the surface, the collected in a sample chamber; a sample chamber that could be imaged directly; a sample chamber whose exposure to an eluent results in the flow of the chemical marker to an imaging zone using microfluidics; a microfluidic system comprising a lateral flow membrane; and a lateral flow membrane characterized as hydrophilic, hydrophobic or super hydrophobic.

In another embodiment, the present invention includes a method of assessing skin condition comprising: collecting a skin sample using an adhesive strip that collects a sample of skin; measuring the intensity level of one or more analytes that interact with the skin sample; and analyzing the contrast levels against the background by imaging the strip comprising the skin sample over time. In one aspect, the adhesive strip comprises a backing and at least two adhesive regions on the backing, wherein the adhesive is selected to capture a surface sample, wherein one or more agents are disposed in the adhesive region capable of interacting with the surface sample to aid in measuring one or more components of the sample. In another aspect, the sample comprises one or more chemical species that interact with the one or more agents. In yet another aspect, the backing comprises a disposable card comprised of cardboard or vinyl and the backing is sized for a cartridge having predetermined dimensions. In another aspect, the adhesive is selected to remove skin cells from the stratum corneum. In another aspect, the performance of a skin product is assessed by sampling skin with an adhesive strip used to collect a sample of skin treated with a product and analyzing the contrast levels against the background by imaging the sample strip over time. In another aspect, the effect of a local environment on skin condition comprising an adhesive strip used to collect a sample of skin and analyzing the contrast levels against the background by imaging the sample strip over time and comparing to a control skin sample.

Another embodiment of the present invention is an adhesive strip comprising an adhesive region for sampling a skin sample, a pressure sensitive dye to assess pressure, and a moisture sensitive pad to assess moisture on the skin.

### BRIEF DESCRIPTION OF THE DRAWINGS

For a more complete understanding of the features and advantages of the present invention, reference is now made to the detailed description of the invention along with the accompanying figures and in which:

FIG. 1 shows mildly green fluorescent epicocconone reacts with nucleophilic amines in proteins to produce a strongly red fluorescent complex;

FIG. 2 shows a carbonylated protein reactive dye would be one that emits in the blue region when excited at 365 nm: 7-diethylaminocoumarin-3-carboxylic acid hydrazide;

FIGS. 3A to 3D show various test strips designed to sample a surface shows here with two or more adhesive regions;

FIG. 4 shows a tape strip of firm but flexible composition would include several separate and diverse regions isolated by thin films;

FIG. 5 shows a tape strip of firm but flexible composition would include several separate and diverse regions isolated by thin films;

FIGS. 6A to 6D show various forms of foldable tape strips designed with a transparent pre-analysis region (hydrogel) on one side of the strip and across the fold a sample region;

FIG. 7 shows the test strip is made of a flexible adhesive and backing or film, such that after sampling the tape strip can be physically manipulated or mechanical pulled;

FIG. 8 shows that multiple attributes listed above are combined into a test strip capable of performing multiple assays;

FIG. 9 shows an example of a unique identification pattern for use with the present invention;

FIG. 10 shows that carbonylated proteins can then be imaged and analyzed the reader system and software program;

FIG. 11 shows one example of a method of care and system using the present invention;

FIG. 12 shows a pre-test selection screen;

FIG. 13 is an example of make-up analysis test assessing amount of coverage; and

FIG. 14 shows an image of an adhesive after sampling, testing, placing in cartridge and reader system and optical interrogation and analysis by software system;

FIG. 15 is a graph of an untreated forehead treated with an exposure to white LEDs at 20 s intervals for 20 minutes;

FIG. 16 are images showing various percentages of hydration of a cheek treated with an exposure to white LEDs at 20 s intervals for 10 minutes;

FIG. 17 is a graph showing the percent hydration of an untreated cheek treated with an exposure to white LEDs at 20 s intervals for 10 minutes;

FIG. 18 is a graph showing the percent hydration of a cheek treated with Aveeno SPF70 treated with an exposure to white LEDs at 20 s intervals for 10 minutes;

FIG. 19 is a graph showing the percent hydration of an untreated forehead treated with an exposure to white LEDs at 20 s intervals for 20 minutes; and

FIG. 20 is a graph showing the percent hydration of a forehead treated with a high-end moisturizer treated with an exposure to white LEDs at 20 s intervals for 20 minutes.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Generally, all technical terms or phrases appearing herein are used as one skilled in the art would understand to be their ordinary meaning.

A variety of unique methods have been discovered and invented to collect and sample biological materials for diagnostic and analytical purposes. This invention is directly related to sampling of biological mediums such as skin and hair by utilizing existing sampling techniques, i.e. adhesive membranes, in combination with novel chemistry assay technology.

The present invention includes the ability to apply novel color-changing, multi-functional and analyte binding reagents and dyes with adhesive sampling methods to achieve an integrated non-invasive skin sampling diagnostic assay.

Processing and Analysis of Surface Samples. Surfaces contain important information in that they are the first barrier to penetration (e.g., skin), they typically reflect the greatest environmental effects, and they contribute strongly to a viewer's aesthetic interpretation. Consequently, numerous sampling techniques have been developed, including those that use adhesive devices, swabs, and absorbent pads. These sampling techniques may also incorporate pre-processing of the surface, pre and post-processing of the sampling device, and several forms of detection and analysis to accentuate desired parameters. The present invention includes new surface sampling techniques and applications, especially for skin and hair. The tape strip or swab themselves become a vehicle for delivery of buffers, reagents, dyes, chelators, and filtration agents, as well as a support surface for the necessary processes and reactions to occur. All these components expose, react, separate and detect chemical and biological markers and analytes that may be correlated to, for example, various skin and hair conditions, treatment regimens, product usage parameters, environmental exposures and various aesthetic and medically relevant parameters.

The present invention includes novel surface sampling devices, techniques, and processes. One aspect of the invention includes the ability to combine a series of sequential modules and separate regions into a singular device for sample processing. The device itself or the results of the processes may be inserted into an automated instrument in such a fashion that optical signatures and patterns may be integrated and corresponded to, via software algorithms and database comparison, both known and presumptive diagnoses and associated recommendations. Non-limiting examples of surface samples include skin and hair samples.

In order to sample, the thin film covering the adhesive region would be removed, the adhesive region applied with proper even pressure to the subject to be sampled, and the tape strip removed with proper even pressure.

As used herein, the term “surface samples” refers to any surface, whether a top surface or layer or surfaces or layers that are exposed subsequent to some form of processing, e.g., scraping, cleaning, abrasion, peeling (mechanical, chemical, etc.). Surfaces are not limited merely to existing surfaces but also include newly derived or exposed surfaces. For example, in the case of multi-tape tape stripping on the same location, with each skin sample removed, the newly exposed skin layer would become the new surface. As another example, both a sample of an apple's exterior as well as a sample of an apple's interior when cut open would be considered surface samples as applied to this invention.

The present invention includes pigments, dyes, or chelators that can be used on surface samples in order to accentuate certain parameters, label analytes, or bind analytes. As required for these applications, buffers, reagents, heating or cooling, and mixing would also be incorporated.

There are many potential areas of use for such a skin and hair analysis system: a) medical spa industry, which offers aesthetic services such as laser-therapy, Botox, chemical peels, hair-removal, etc.; b) salons, spas, and resorts that offer products and treatments such as facials, wraps, peels, and full body treatments, etc.; and c) health & wellness specialists that tender homeopathy, naturopathy, chiropractic, and herbal medicine; d) dermatologists; e) aestheticians; and f) pharmacy retailers (compounding or retail chains). The present invention provides health care professionals information about the consumer/client/patient's skin or hair that will be useful in choosing the appropriate skin or hair care products to remedy the condition or improve quality of skin and health. Another potential area the skin and hair analysis system would be useful is at the beauty counter of high-end retailers and department stores where personal care and cosmetic products are sold. The present invention may also be useful to industry, clinical research companies, and ingredient manufacturers. Most notably, the consumer will benefit.

Current methods for analyzing skin are: 1) imaging or 2) determination of physical factors. For example, current methods for determining appearance of skin, fine lines, wrinkles, ageing, sagging and UV damage are mostly visualization of the face by various imaging apparatus. Visual images range from a basic or magnified photograph to three-dimensional (3-D) optically enhanced images. Most of these images can be self-assessed or graded visually by an expert during a consultation in salons, medical spas or by dermatologists. Scopes and sensors are the most widely used imaging devices. Typically, these systems consist of a camera that magnifies skin and pores, and a sensor that measures oil, hydration and elasticity of the skin. Commercial examples are the étude and i-scopeUSB made by the company Moritex. Skin appearance, oiliness, dryness, elasticity, texture, pigmentation, and squames are analyzed.

Another example is 3-D imaging, which is more expensive but not much more sophisticated in terms of information provided to the customer/client/patient. Spectral imaging detects melanin and hemoglobin under UV or fluorescent lighting. A software tool and statistical model interpret the information to generate a report that evaluates wrinkles, spots, pore size, skin tone (evenness), bacterial content and UV spots indicative of sun damage or oxidation. Examples of commercially available systems are the VISIA by Canfield Imaging Systems or the ClarityPro by Moritex. There are several problems associated with these imaging systems, such as lighting and positioning. A shift in position or slight variations in lighting may cause differences that appear to be skin conditions, but are not. The present invention overcomes these problems because the skin sample is taken from the person and inserted into the USB or reader device so that lighting is consistent and correctly positioning the face is irrelevant.

Various other methods exist to determine physical factors such as skin hydration, elasticity, and barrier function. Skin hygrometers are type of apparatus that measure electrical capacitance and conductance of skin in order to determine the skin's relative hydration. Hydration has also been measured via spectroscopy, including acoustic, infrared, and Nuclear Magnetic Resonance (NMR), and corneometry. Skin elasticity is often measured using a ballistometer, dermal torque meter, or by pinch recoil. Skin barrier function and water evaporation are usually measured by transepidermal water loss (TEWL). Most of these methods for evaluating skin parameters are used in clinical efficacy trials and would not work at beauty counter in a retail distributor or in the medical spa or salon. The present invention provides for such application. The user can determine efficacy of particular ingredients and correlate results to definite biomarkers.

Current state-of-art is that skin type is generally described by purely physical factors such as dry, oily, normal or combination skin. Combination skin encompasses both oily and dry skin patches. Current methods for determining skin type range from customers completing inclusive questionnaires to non-invasive methods. Questionnaires are typically used at the counter in department stores or for online product sales. For example, companies such as Chanel, Clinique and Olay use questionnaires to assess customer skin type and recommend skin care products and cosmetics based on those skin types. Consumers answer questions about skin sensitivity to sunlight; skin color (fair, light, medium, dark, yellow, pink, etc.); eye and hair color; pore size; breakout tendency or if acne-prone; oily versus dry; visible wrinkles; and so forth. The answers to these questions define skin characteristics and help the consumer chose appropriate products.

Currently available methods for determining skin condition do not provide adequate information to determine the cause of skin conditions. This approach is very subjective and relies on the customer's answers. Preconceived notions and inaccurate information will vary the answers and are not very helpful in assessing actual conditions. Most of the skin conditions or biomarker are non-visible and cannot be determined readily by visual assessment or answering a few subjective questions. The present invention reveals the underlying biomarkers and the causes of skin conditions or problems, which provides for specific personal care. For instance, red skin can be caused by many different conditions. Dry skin, itchy skin and flakiness are all symptoms of several different problems or diseases that could be caused by ceramide breakdown, loss of natural moisturizing factors, irritants, soap, allergies, bacteria, oxidation, sun damage and/or eczema, psoriasis, etc. Currently, consumers are forced use products by trial and error until something works.

Tape-stripping is a non-invasive approach that permits a direct quantitative and qualitative assessment of biomarkers from the skin surface and stratum corneum (SC). Examples of tape-stripping products commercially available are D-Squame (CuDerm), Sebutape (CuDerm), various adhesive and mailing tapes (3M), and cyanoacrylate resin. Samples are taken by applying the adhesive tape to a target area of skin in a manner sufficient to isolate an epidermal sample adhering to the adhesive tape. Layers of the SC can be sequentially removed by repeated application of pieces of adhesive tape. The epidermal sample contains biomarkers that correlate to skin conditions. Currently, tape-stripping is used in research or for marketing.

These methods give varying degrees of qualitative information, but do not offer any way of detecting specific biomarkers or skin analytes linked to various skin conditions. For instance, an apparatus can determine that skin is dry or less elastic. However, the apparatus does not give the consumer a reason for the dryness or loss of elasticity. It is necessary to determine various biomarkers that correlate to skin conditions in order to make such a determination. The present invention correlates biological markers to skin or hair conditions.

The current invention detects biomarkers that correlate to specific skin conditions and provides quantitative data for skin assessments unlike 3-D imaging machines, sensors, and scopes. Consumers, salon professionals, medical spa professionals, and personal care product manufacturers are in need of consumer/product feedback to ensure best product usage, compliance and effectiveness. The current invention provides information to the consumer which enables more product specialization and better product selection. Less wasteful spending on product trial-and-error is inevitable as customers are specifically determined or diagnosed according to biological molecules rather than superficial surveys or general physical qualities. This will ultimately improve consumer confidence in a product, brand support for manufacturers, and improve skin health.

Abundance of biomarkers correlating to skin conditions exemplify why it is currently difficult to coordinate products to consumer skin conditions. Stratum corneum is the outer layer of the skin that interacts with the environment. Biomarkers naturally occurring in the stratum corneum are natural moisturizing factors (NMFs), proteins, enzymes, lipids, fatty acids, ceramides, and cytokines. The presence of aldehydes, carbonyl proteins, vitamins, surfactants, metals, pollutants, porphyrins, and bacteria are indicative of various skin conditions. Imbalance of naturally occurring biomarkers or the presence of one or more analytes correlate to skin conditions including, but not limited to dryness, itchiness, flaking, scaling, roughness, wrinkles, elasticity, age spots, bumps, redness, and inflammation. These skin conditions are implicated in several skin problems or diseases, such as oxidative or sun-damage, dehydration, acne, irritation, aging, wrinkles, inflammation, rosacea, eczema, psoriasis, and allergic or contact dermatitis.

Natural moisturizing factors molecules are generated by hydrolysis of the protein filaggrin into free amino acids (serine, glycine, arginine, ornithine, citrulline, alanine, histidine), urocanic acid, pyrrolidone carboxylic acid, lactate, sugars, urea, chloride, sodium, potassium, ammonia, uric acid, glucosamine, creatine, calcium, magnesium, phosphate, citrate and formate. NMFs are implicated in skin conditions such as dryness, flaking, scaling, inflammation, and ageing.

Numerous proteins are found in stratum corneum, such as keratin, corneodesmosin, loricrin, suprabasin, desmoglein, and others. It is further well known in the art that oxidative stress causes: 1) oxidative cleavage of proteins; 2) direct oxidation of amino acids; 3) carbonyl groups introduced into proteins via reactions with aldehydes derived from degradation of lipid peroxides. Increased carbonyl protein levels correlate to dryness, scaling, roughness, wrinkles, loss of elasticity, and ageing. Furthermore, aldehydes in cigarette smoke cause damaging carbonyl formation in skin.

Vitamins, derivatives, forms and complexes. UV exposure and oxidation cause a decrease in the human SC's natural anti-oxidants such as vitamins A, C, and E (in various derivative, forms and complexes). The major form of vitamin A is an alcohol (retinol), but can also exist as an aldehyde (retinal), as an acid (retinoic acid), as an ester (retinyl palmitate) and as beta-carotene. Vitamin A is known to improve condition of skin; but retinol causes inflammation of the skin. Vitamin C (in its various derivatives, forms and complexes) is an anti-oxidant that also enhances the synthesis of collagen. Vitamin B3 (Niacin/Niacinamide) helps the skin retain moisture and upregulates ceramide synthesis. Vitamin D deficiency may occur with use of sunscreen because sunlight is necessary to convert Vitamin D into a bioavailable form. Vitamin D3 is produced photochemically in the skin from 7-dehydrocholesterol. Vitamin K is known to repair dark, under eye circles and bruises as well as healing spider veins.

Enzymes found in the stratum corneum include, but are not limited to beta-glucocerebrosidase, phospholipases, acid phosphatase, serine proteases: trypsin (chymotrypsin), cholesterol sulfatase, sphingomyelin deacylase, prosaposin, transglutaminase, peptide methionine sulfoxide reductases, and acid ceramidase. Phospholipases: Type IV cPLA(2)-α (calcium dependent) and type I or II sPLA(2) (secretory) are found in the skin. Type II sPLA(2) is implicated in inflammation. Increased acid phosphatase activity correlates to dry, itchy skin. Reduced trypsin activity correlates to dry, itchy skin and scaling. Altered levels of prosaposin, a regulator of sphingolipid metabolism, are implicated in dry, itchy skin as well as roughness, bumps and inflammation. Peptide methionine-S-sulfoxide reductase is a unique repair enzyme indicative of skin-oxidation and cell-ageing.

Cholesterol esters and cholesterol sulfate are part of the stratum corneum barrier function. Cholesterol sulfate accumulates when deficient in steroid sulfatase enzyme (recessive X-linked ichthyosis—genetic disease); induces transcription of transglutaminase; inhibits serine proteases involved in desquamation. Transglutaminase activity correlates to dryness and scaling of the skin.

Many analytes or biomarkers interact with each other in various synthesis and degradation pathways. For example, Ceramide EOS (Cer(OS)) main ceramide component of stratum corneum. It contains an omega-hydroxy fatty acid ester-linked to linoleic acid and amide-linked to sphingosine. Free linoleic acid is necessary to maintain skin barrier function, and as such altered levels correlate to dry skin, scaling and inflammation. Furthermore, decreased levels of free sphingosine reflect decreased levels of ceramide and diminished acid ceramidase activity which cause scaling of the skin. Another example, a decrease in CER(EOS) levels causes an increase in sphingomyelin deacylase, which competes with sphingomyelinase for the ceramide precursor sphingomyelin, causing an increase in sphingosyl phosphoryl choline. Sphingosyl phosphoryl choline stimulates proliferation and up-regulation of plasminogen activator. Elevated levels of sphingomyelin deacylase and sphingosyl phosphoryl choline correlate to dry, itchy skin as well as roughness, bumps, and inflammation.

The presence of unusual species are indicators of skin conditions. Presence of ω-hydroxy acid, stimulates ceramide production in the epidermis, and can be correlated to scaling and inflammation. Ceramide(AS) is an unusual species and is correlated to dry, itchy, scaling, roughness, bumps and inflammation. Triglycerides, short-chain saturated fatty acids and unsaturated fatty acids are sebaceous contaminants whose presence may serve to disrupt barrier organization at skin surface correlated to dry skin. Phospholipids should not be present in healthy stratum corneum.

Cytokines are known to cause wrinkles, redness, and inflammation. Several interleukins have been detected on the skin surface. For example: IL-8, IL-6, IFN-γ, IL-4, IL-13 cause inflammation; TNF-α correlates to scaling, roughness, redness, inflammation (Benson, et al., 2006); and IL-1α and IL-1RA (receptor antagonist) involved in epidermal signaling and indicative of ageing and inflammation. Glucocorticoids delay barrier recovery and lead to dry, itchy skin.

Surfactants are known to bind to stratum corneum proteins and cause dry, itchy skin, scaling, roughness, loss of elasticity, bumps, and inflammation. They are usually used in soaps, syndets, and detergents. Sodium lauryl sulfate (SLS)/sodium dodecyl sulfate (SDS) and sodium lauroyl ether sulfate (SLES) are anionic surfactants and bind proteins of the SC. Sodium lauroyl isethionate (SLI) is also anionic but binds ⅕ as strongly to SC proteins as SLS/SDS. Lauroyl amido propyl betaine is amphoteric and binds SC proteins to a much lesser degree than anionic surfactants. Other surfactants known to bind SC proteins are monoalkyl phosphate, sodium cocoyl isethionate, cocamidopropyl betaine (CAPB), and alkyl polyglucoside (APG).

Analytes: Metals such as nickel are irritants that can cause bumps, redness and irritation. (Nyren, Kuzmina, & Emtestam, 2003)

Acne is caused by various factors, including excessive sebum and poor desqammation of the stratum corneum. Indicators are bacterial contamination (P. acnes) and porphryins secreted by bacteria (coproporphyrin I, coproporphyrin III, and protoporphyrin). These are metal-free fluorescent porphyrins that can be easily detected on the skin surface.

Hormones: Dihydrotestosterone (DHT) is a hormone that has also been correlated to oily skin, bumps, redness, inflammation. A decrease in the hormone estrogen causes dryness and wrinkles. This condition often occurs during the aging process.

Biomarkers, Epidermis, Dermis, etc. Samples from a tissue can be isolated by any number of means well known in the art. Invasive methods for isolating a sample include the use of needles, for example during blood sampling, as well as biopsies of various tissues.

There are biomarkers in epidermis, dermis or other tissue samples that correlate to skin conditions and diseases. For example, matrix metalloproteinases (MMPs) and their inhibitors (MMPIs) are highly regulated molecules found in the dermal layer. MMPs are known to play a role in pathological conditions such as inflammation and wound healing. There are several families of MMPs, one group are called collagenases (MMP-1, MMP-8, and MMP-13), which can cleave interstitial collagens I, II, and III at a specific site as well as degrade other ECM and non-ECM molecules. Specifically, MMP-1 is known to degrade collagen I, collagen II, collagen III, gelatin, and proteoglycans. MMP-8 is known to degrade collagens I, II, III, V, VII, IX, and gelatin. MMP-13 in known to degrade collagens I, II, III, IV, IX, X, XIV, fibronectin, and gelatin. The presence of certain MMPs and MMPIs, as well as variations in basal level can be biomarkers correlated to aging and an increase in wrinkles and roughness as well as a loss of elasticity.

Similarly, the presence and relative levels of glycosaminoglycans (GAG) and proteoglycans found in the dermal layer can be correlated to the roughness and elasticity of the skin. Hyaluronan is found in varying biological forms both in the epidermal and dermal layers of the skin and can be correlated to wrinkling of the skin. Biglycan, decorin, and fibronectin play a significant role in roughness, wrinkles, and ageing of the skin.

Molecules that form the core structure of the dermal layer, such as elastin and collagen are major players in ageing of the skin, leading to roughness, wrinkles, and loss of elasticity.

The present invention is used to obtain quantitative data as well as qualitative imaging; broad spectrum of test measures, such as novel biochemical assays, measure custom markers by product, and design by skin condition; product performance indications; product selection information for consumer/product matching with a quick; simple system; and at a lower cost than 3-D imaging.

By identifying non-visible skin health markers, more information is given to the professional and consumer enabling more product sales, more product specialization, improving consumer confidence in a product, improving skin health and increasing brand support for manufacturers.

In one embodiment, the invention provides a method enabling analysis of skin and hair samples of a person, the method including a step of taking a skin or hair sample. A chemical reagent for identification of specific components in the sample may be added. At least one image is taken with one or more light sources; and non-visible spectrum light captures the image electronically. A memory device will store the image, which can be analyzed and displayed immediately or stored for later processing and display. In one embodiment, the present invention comprises a reader device, disposable test trips or cartridges, and a computer-implemented system to provide a product feedback method.

Skin samples are taken by tape-stripping method and incorporated into a carrier such as a cartridge or test strip. Cartridges or test strips will detect various analytes or biomarkers that correlate with various skin conditions, including but not limited to:
1) aldehydes, carbonyl proteins, and decreases in vitamin E levels are indicative of skin oxidation and products containing anti-oxidants, sun protection, vitamins should be recommended;
2) a depletion in NMF, ceramides, and varying levels of skin surface enzymes are indicative of dry skin and products such as moisturizers, soaps, and ceramide production enhancers should be recommended;
3) the presence of porphyrins or excessive sebum (oil) coupled with poor desquamation are indicative of acne and products containing salicylic acid, benzoyl peroxide, Retin-A along with specific skin care regimens should be recommended;
4) surfactants, metals (Ni²⁺), pollutants and allergens can cause redness or irritation of the skin and the appropriate chemical and natural peels, masks, and detoxification products should be recommended;
5) ceramides, carbonyls, aldehydes, and collagen levels all relate to skin aging and wrinkles and products with collagen-enhancing treatments, peptides, sunscreen, anti-oxidants, Botox, or surgery should be recommended; and
6) new analytes, specifically requested analytes, or ingredients will be incorporated into new cartridges (can correlate analyte to new products being developed for research and product feedback).

At least one or more of these cartridges are inserted into a reader device. Images are captured of the skin sample. Another embodiment is to incorporate chemicals into the cartridge or test strip that will react with the skin sample. The software will have algorithms to correlate biomarkers in the skin sample with skin conditions. A report will be generated for the professional or consumer who can then recommend various products relating to the skin condition.

UVA and UVB sunrays, peroxide attack, Michael or Schiff reactions with aldehydes resulting from lipid peroxidation, and other oxidative mechanisms contribute to the oxidation of proteins, to produce carbonylated proteins, in the stratus corneum. Such oxidation can interfere or impede the performance of the protein, eventually contributing to premature ageing, dehydration, and general unhealthiness of the stratus corneum. For this reason, cosmetic research and development as well as choice of cosmetic products must consider the level of carbonylated proteins. A good reference is the ratio of carbonylated to total protein.

An efficient analysis of stratus corneum samples for this ratio therefore becomes necessary. In the case of a non-invasive adhesive sample, for instance, a tape strip, simultaneous analysis of the layer of skin removed for carbonylated proteins versus total proteins would minimize error due to sampling variations, such as location of the tape strip on the skin, layer of skin, application pressure on the tape strip, removal speed and angle, etcetera. For efficiency, the number of reaction or loading steps and washes should be minimized. A fluorescence assay fulfills these requirements, along with giving nanogram sensitivity necessary to such a small amount of sample.

Carbonylated Protein Assays. Since the carbonyls in carbonylated proteins are frequently aldehydes and ketones, an amine linked to a fluorophore as a Schiff reagent becomes the obvious choice for a carbonylated protein assay. The linkage typically involves a hydrazide, semicarbazide, carbohydrazide, and thiosemicarbazides, although sometimes aniline-based fluorophores suffice. Some examples of these dyes are fluorescein-5-thiosemicarbazide, 7-diethylaminocoumarin-3-carboxylic acid hydrazide, Texas Red hydrazide, and 7-amino-4 methylcoumarin. To perform this assay, the adhered sample is immersed in the reactive dye buffer until reaction is complete. None of these fluoropohores is non-fluorescent prior to reaction nor do they significantly shift in emission wavelength upon reaction, so a wash step, typically with phosphate buffered saline, must ensue.

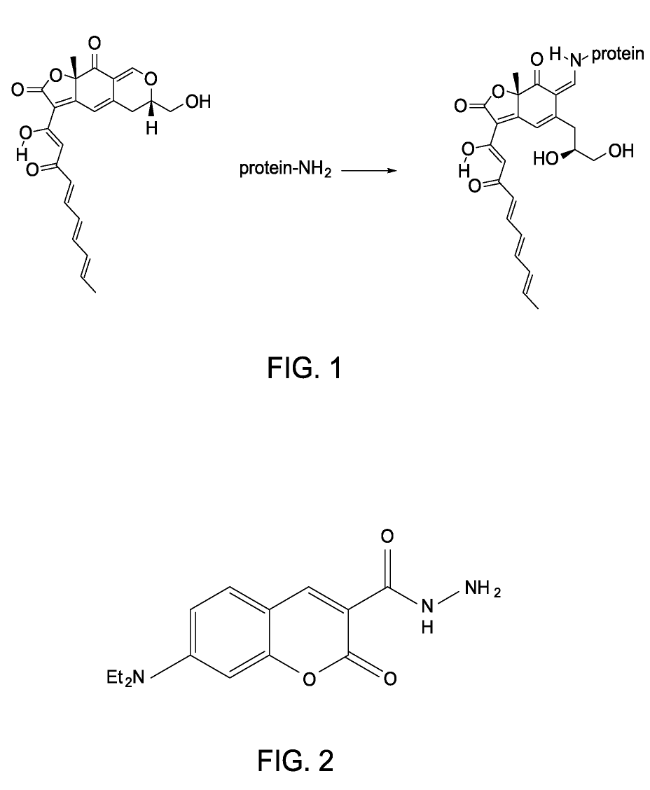

The total protein fluorescent assay of choice FluoroProfile, supplied by Sigma-Aldrich, results from the reversible reaction between virtually non-fluorescent epicocconone with nucleophilic amines on the protein to yield a red-orange emitting fluorophore with an excitation maximum at 390 nm and an emission maximum at 605 nm (FIG. 1). The FluoroProfile assay exhibits a linear range of 40 ng to 200 ug per milliliter of protein and a coefficient of variance among different proteins of 16%, as compared to 11% for the less sensitive, smaller linear range colorimetric BCA assay. To perform the assay, the adhered sample is simply immersed in the epicocconone buffer until reaction is complete. No washing step is necessary. FIG. 1 shows mildly green fluorescent epicocconone reacts with nucleophilic amines in proteins to produce a strongly red fluorescent complex.

Other total protein fluorescent assays include non-fluorescent fluorescamine and o-phthaldehyde. Again, nucleophilic amines in the protein react to produce fluorophores. These two assays exhibit a greater coefficient of variance among proteins as well as a smaller dynamic range, when compared to FluoroProfile.

Due to the interactivity of epicocconone and fluorescent hydrazides and to the FluoroProfile's assay not needing a rinse, the logical order of the assays follows. First is the carbonylated protein assay, a rinse, and then the total protein assay. Simultaneous imaging demands that both assays' fluorophores be excited at the same wavelength. For a UV LED excitation of 365 nm, the total protein epicocconone protein complex emits at 605 nm. To ensure as little spectral overlap as possible, one non-limiting choice for the carbonylated protein reactive dye would be one that emits in the blue region when excited at 365 nm, e.g., 7-diethylaminocoumarin-3-carboxylic acid hydrazide (FIG. 2), which emits at 468 nm. Performing both assays on the adhered samples, quantifying the amount of blue and red fluorescence by image analysis, correcting for spectral overlap and other interferences, and taking a ratio of the corrected blue over corrected red yields the oxidized to total protein ratio.

1. Functionalized Tape Strips.

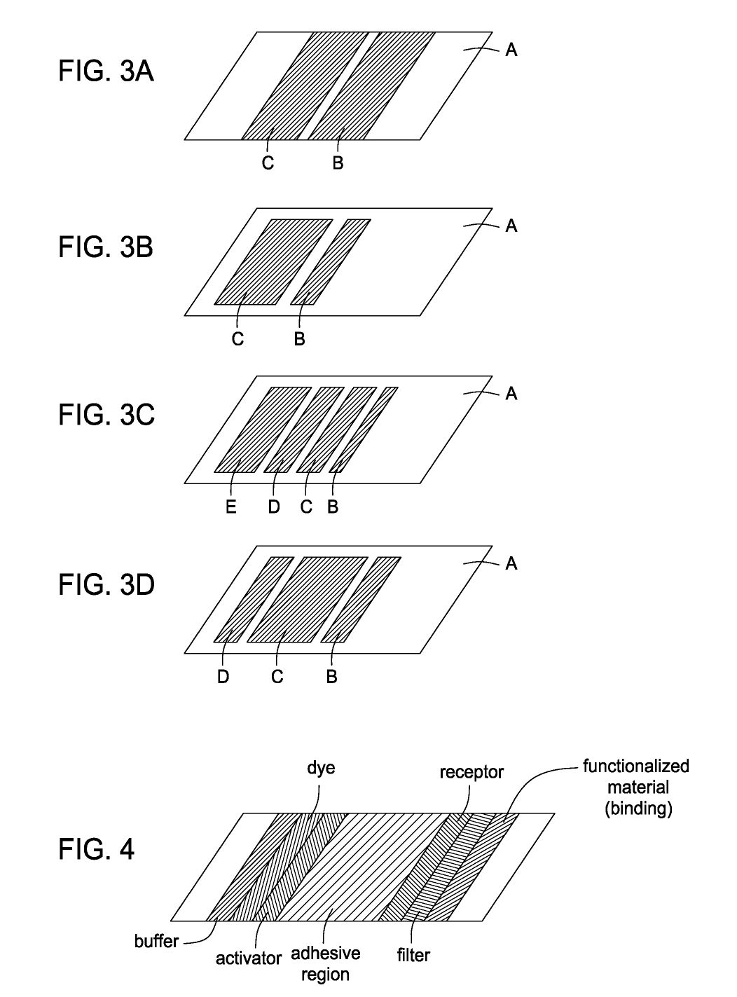

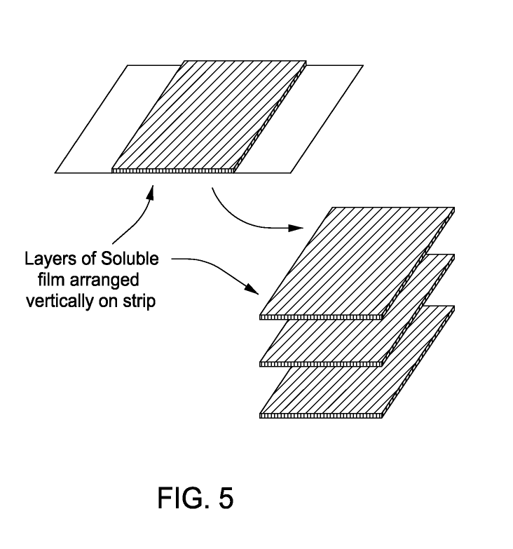

In one embodiment, a test strip was designed as a sampling device with two or more adhesive regions (FIG. 3 A-D). A backing (A) is depicted that includes two adhesive regions (B, C). In one embodiment the adhesive regions (B, C) are the same size and evenly spaced on the opposite sides of the mid-point of the backing or not evenly spaced and having varying sizes, or a plurality of adhesive regions ((B, C, D, E) or various sizes (B, C, D). Also, each of the adhesive regions may also include one or more functional agents that aid in the detection or visualization of one or more components of the surface sample. These may have varying adhesive qualities such that upon use the sampling device gathers different amounts of sample in distinct regions. These may also have varying adhesives compositions such that functionalities beyond adhesion and sample collection are provided for. These adhesive compositions serve as both a collection surface and a delivery mechanism for novel chemistries that may alter or interact with the sample or analytes present in the sample. Some regions may in fact not comprise adhesive compositions but rather materials designed to carry reagents, dyes, or synthetics receptors that would be release upon the appropriate signal, time, or conditions.

In further embodiments these adhesives regions may also include differing adhesive compositions such that different regions offer different functionalities in addition to sample collection. An in-exhaustive list of composition classes that can be utilized to this end include: colored adhesives, adhesives comprising natural adhesives synthetic adhesives, drying adhesives, contact adhesives thermoplastic adhesives, reactive adhesives, UV and light curing adhesives or pressure sensitive adhesives.

The adhesives physical properties are optimized for optical imaging, illuminated from LED's in the IR, visible and UV wavelengths.

In one embodiment, a tape strip of firm but flexible composition would include several separate and diverse regions isolated by thin films (FIGS. 4 and 5). FIG. 4 shows a backing that may include one or more regions, e.g., an adhesive region, a region that includes as an active agent a receptor, an activator, a filter, a dye, a buffer, an a functionalized material (e.g., an agent that binds to one or more components suspected of being in the surface sample. The most obvious region is that of the adhesive that is applied to a surface such as skin to remove a sample.

FIG. 5 shows another embodiment in which layers of soluble films are arranged vertically on the backing or strip. Other regions would comprise buffers, reagents, dyes or pigments, filters, and chelators. In most embodiments, the components of each region would be solids, gums, or hydrogels that would be fixed into place by the thin films. The components could be the necessary compounds themselves, solutions of the compounds that had been evaporated or lyophilized to solids or gums, or hydrogels that incorporate the compounds. The regions on the tape strip would typically be separate, as necessary to prevent premature mixing, with perhaps the most mutually sensitive regions as far apart as possible on the strip. The surface area of the region would consider ease of manufacture, concentration of reagent, and solubility of the reagent. In the simplest conformation, the regions would be parallel to the adhesive region (FIG. 4). However, another conformation adopting vertical separation of regions via soluble films (FIG. 5) could be advantageous when a region must be exposed in a particular order or with a particular time delay.

After sampling the tape strip would be introduced into a processing chamber. This chamber would be sealed, and a solvent or buffer could be introduced via syringe or blister pack, or if it were not already incorporated in the processing chamber, either non-sequestered or sequestered in a breakable or soluble enclosure, such as a capsule. Upon exposure to the correct solvent, the thin films would either dissolve or dislocate, and the components of each region could dissolve or dislocate. Mixing could be accomplished by shaking, vortexing, sonnicating, pumping, and/or heating. The extent of mixing and/or heating would ensure complete dissolution and reaction. A sequential or quenching solvent, buffer, or reagent could be introduced via syringe, blister pack, or delayed release of a previously incorporated capsule within the same processing chamber.

In an alternative embodiment, the tape strip with the surface sample is inserted into a processing chamber which incorporates buffers, dyes or pigments, reagents, chelators, and filters as solids or liquids in separate regions or compartments (Figure) not incorporated into the tape itself. If regions, the solids would be reversibly adhered to the inner surface of the chamber, typically under a soluble film, preferably with the most cross-reactive reagents spatially separated the most. If compartments, the solids or liquids would be retained within breakable or soluble enclosures, such as capsules. The chamber could also contain a solvent, buffer, or solution, either non-sequestered or sequestered in a breakable or soluble enclosure. Upon insertion of the tape strip, the chamber would be sealed, and a solvent or buffer can be introduced via syringe or blister pack. Mixing of the components may be accomplished by shaking, vortexing, sonnicating, pumping, and/or heating. The extent of mixing and/or heating would ensure complete dissolution and reaction. A sequential or quenching solvent, buffer, or reagent could be introduced via syringe, blister pack, or delayed release of a previously incorporated capsule within the same processing chamber.

Several different end products from the processing chamber could result. If buffers and solvents are chosen such that the sample remains on the adhesive region, the tape strip may simply be removed and if necessary rinsed and/or shake, pat, or air-dried. It would then be appropriately introduced into the detector and analyzed.

If the sample, and especially the analyte, dissolves, the solvent, or a dilution thereof, may be introduced into the detector and analyzed. The solution may also be further processed by exposing it to a filter, lateral flow device, or chromatographic material, such as a thin-layer chromatography strip or a solid-phase extraction cartridge. The result of such a separation would then be introduced to the detector and analyzed.

Alternatively and in conjunction with above descriptions of tape strip layout and adhesive compositions, the backing and other material contained can be functionalized to improve other parameters that augment processes such as imaging and optical interrogation or physical manipulation of the sample collected. These include but are not limited to: thermochromic background, tropochromic background, fluorescent background, translucent/transparent background, electrochromatic background, solvochromic background, reflective background, phosphorescent background. Assays surfaces include membranes recognized by one skilled in the art including the compositions nitrocellulose, UVPE, PVDF, hydrophobic membranes known to those skilled in the art of immunosorbent assays.

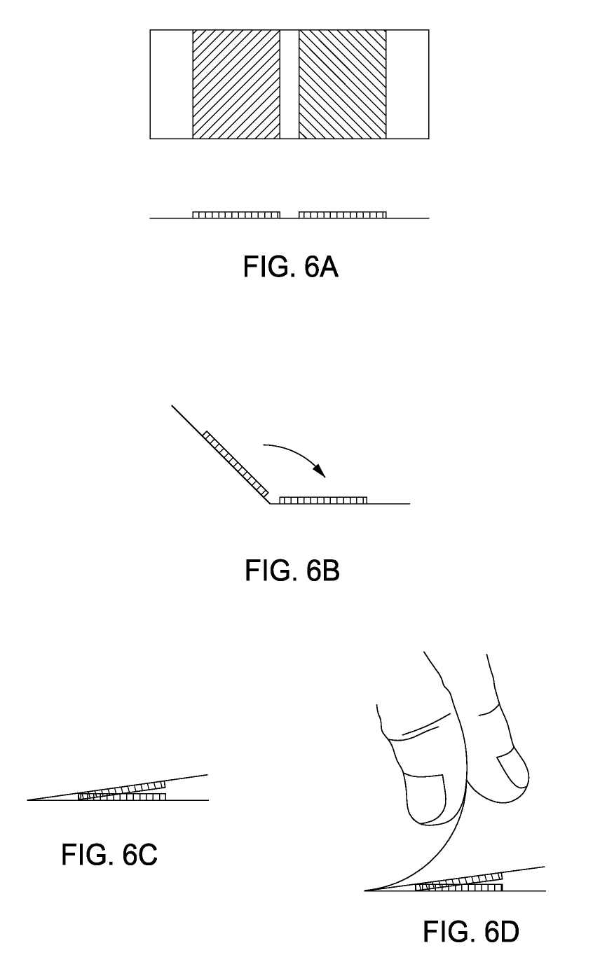

In another embodiment (FIG. 6), a foldable backing or tape strip is shown with a transparent pre-analysis region (hydrogel) on one side of the strip and across the fold a sample region (6-A). The sample is collected the tape strip is designed to be folded over, bringing the hydrogel portion into contact with the sample thereby activating a chemical composition (6-B-C). The gel component could be any gel capable of carrying reagents and releasing them upon exposure to a solvent. The reagents would be selected for their ability to detect an analyte of interest in the sample on the adhesive portion. The portion of the test strip carry the gel component is removable or contains a transparent backing for imaging (D).

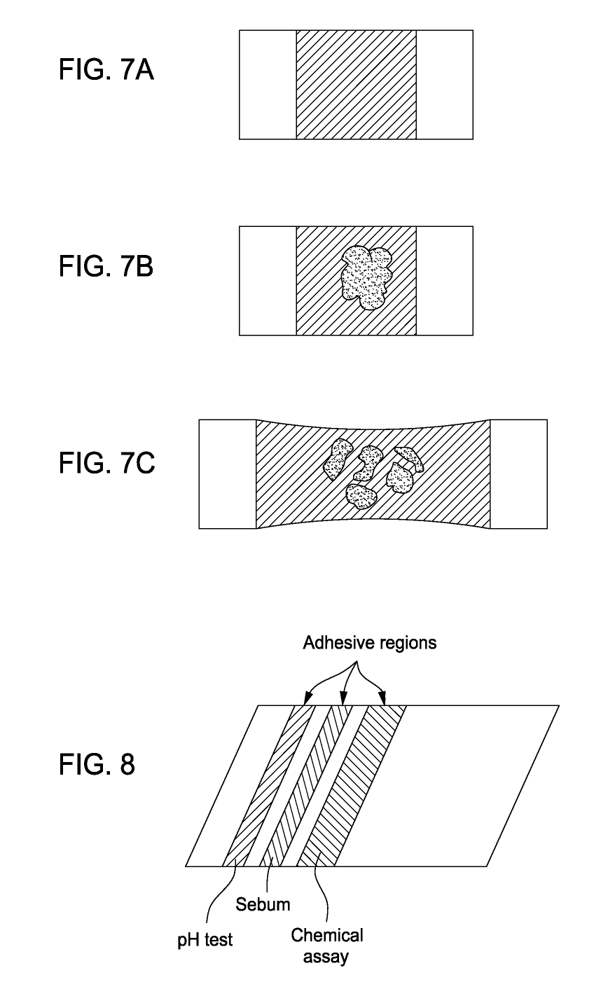

FIG. 7 shows another embodiment the test strip is made of a flexible adhesive and backing or film, such that after sampling the tape strip can be physically manipulated or mechanical pulled, for example in an expandable cartridge fasted to the tape strip, to expand the sample area and cause differential separation of the sampled material to indicate various qualities. Depending of the physical characteristics the skin sample will separate and break apart in different forms and frequency (FIG. 7 A-C).

An adhesive composition designed to collect the mirror image of what it touches in 3D. In one embodiment an adhesive composition is preloaded with an analyte specific reagent, such as a synthetic receptor. Companies such as Beacon Sciences rationally design and synthesize synthetic receptors that provide one-step colorimetric detection of a specific molecule. These compounds are robust such that incorporation into an adhesive composition could provide a functionalized tape strip.

FIG. 8 shows another embodiment with multiple attributes (above) that are combined into a test strip capable of performing multiple assays. In one specific embodiment multiple adhesive regions as shown above are designed for distinct tests. One adhesive region is composed of a ph sensitive adhesive formulation, the next contains a thin film capable of absorbing sebum, the next a chemically sensitive adhesive for a marker of interest. Identifying marks may also be placed on the strip, as shown in FIG. 8.

In another embodiment test strip device containing an MIP adhesive thin film is sandwiched beneath a backing material and an adhesive for removing tissue. In another embodiment the adhesive itself contains a MIP enabling detection of a specific analyte contacted with the adhesive and potential release of a secondary functional molecule such as a dye.

In another embodiment a chemical composition contained in an adhesive surface is activated to do the mechanical shear of removing a protective file.

In another embodiment, a wicking agent is embedded in the adhesive composition. In one embodiment, the tape strip would incorporate a pre-processing module, the sampling module, a post-processing module, and a detection module. These modules may exist in all possible permutations, i.e. entirely separate, overlapping, coexisting, or some modules omitted. The tape strip itself could consist of a wicking membrane, such that after sampling a surface, the initiating end of the tape strip would be immersed in a solvent or buffer that would travel up the membrane to each modular region. As the liquid passes through the pre-processing module, it will dissolve or carry dyes, buffers, reagents, or chelators therein contained and move them into contact with the surface sample in the sampling module. Vital analytes or signaling agents would proceed further onto the post-processing module, which could also contain dyes, buffers, reagents, or chelators, as well as filtration agents. Finally, the liquid would carry the analytes in their detectable form to a detection module with properties to enhance contrast and detection. Again, certain modules can overlap, coexist, or not exist. For example, if a buffer were to move up the tape strip and dissolve a dye that would stain the sample adhered to the sampling module, with free dye and buffer moving on past the sampling module, detection could be performed on the adhered sample, and thus the sampling module would also be considered the detection module (see, e.g., FIG. 6). In another embodiment backing material is chosen for its wicking or lateral flow capacity.

In another embodiment a chemiluminescent background is in one state inactive and later activated. In the inactive state a peroxide film or wax like material is essential solid and separate from a composition of florophore and oxalate embedded in the adjacent layer of the tape strip. Upon heating the peroxide triggers chemiluminesence in the background of the sample zone providing a contrast with the sample, e.g., a temperature dependent amorphous coating.

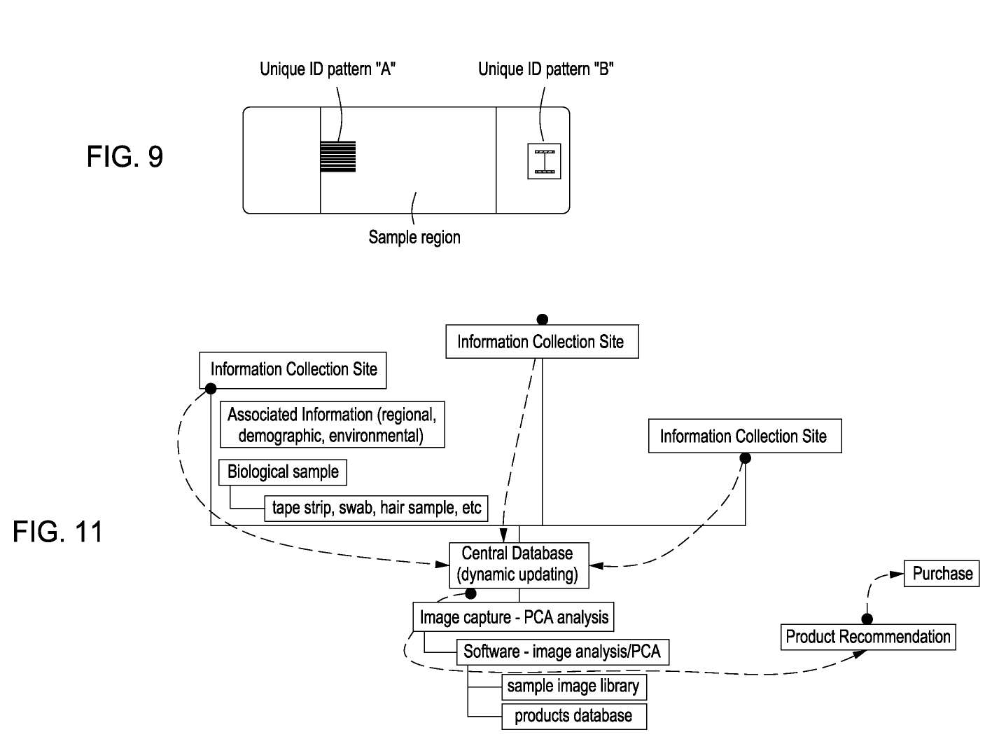

Further embodiments provide for authentication of test strips presented into reader system. In one embodiment a tape composition includes a background with a random colored pattern for security, calibration and test validation interpretable by a software algorithm that processes the digital signal from a camera. In another embodiment an adhesive region with an optical barcode, a decal, or another printed unique identification pattern is interrogated by the imaging system as a method to inhibit naked-eye viewing or to prevent reading by a different imaging system (i.e. requiring the user to use the automated instrument) FIG. 9.

In another embodiment a fluorescent dye is applied to the skin with a sponge like stamp. A tape strip is applied to the pre-treated area and the treated stratum corneum is removed for subsequent imaging and analysis.

In another embodiment a reagent composition containing an indicator molecule is applied to the skin with a sponge like stamp. A tape strip is applied to the pre-treated area and the treated stratum corneum is removed for subsequent imaging and analysis.

In one embodiment an adhesive system contained on a tape strip comprising 7-diethylaminocoumarin-3-carboxylic acid hydrazide and epicocconone is used to collect a skin sample and carbonylated vs. total protein is indicated and imaged to measure to skin damage and oxidization. In one example, a tape strip contains an adhesive region and two more separate dye containing regions, laid out as shown in FIG. 3-D. A first composition on the test strip in area D for example, is a thin film comprising 7-diethylaminocoumarin-3-carboxylic acid hydrazide. This thin film can be designed to be soluble in aqueous buffer. Other release paradigms involve a thermo-sensitive film such that an onboard exothermic reaction zone or external heating source dissolves and releases the dye in the film. Upon release 7-diethylaminocoumarin-3-carboxylic acid hydrazide binds to the total protein content contained in the adhesive sample zone (area B in FIG. 3-D). After a rinse buffer such as phosphate buffered saline is passed over, a second reagent containing thin film on the adhesive (area C) is dissolved to, perhaps by the PBS passed over in the rinse step or other means, releasing the epicocconone that specifically binds to carbonylated proteins in the adhesive sample zone (area B). The test strip is then imaged to capture a carbonylated vs. total protein measurement to indicate skin damage and oxidation.

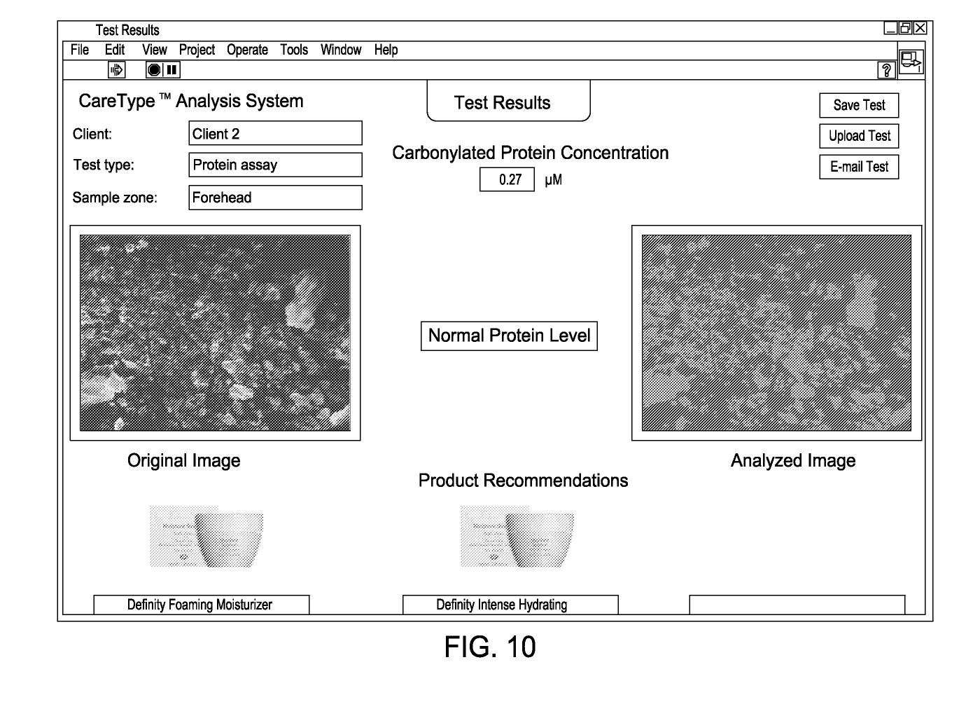

The release of the staining dyes could be accomplished through many different variations according the above listed tape strip arrangements, materials, and compositions provided it meets the following parameters: 7-diethylaminocoumarin-3-carboxylic acid hydrazide is first passed over the sample zone at pH 5.5. The excess 7-diethylaminocoumarin-3-carboxylic acid hydrazide is the rinsed away, and the epicocconone is then passed over the sample zone. Carbonylated proteins can then be imaged and analyzed the reader system and software program as show in FIG. 10. FIG. 10 shows a carbonylated protein assay using fluorescent imaging.

2. Cartridge and device that accepts samples and presents them to an optical imaging device.

This study shows an integrated self-contained reconfigurable diagnostic device for the purpose of applying and adapting solid and liquid phase chemistry assays to samples collected from skin, hair and associated body tissue and fluids.

This study combines existing sampling methods such as swabs, tape strips, absorbent pads and the like with a unique cartridge designed to deliver, expose, react, separate, and detect chemical and biological markers and analytes that may be correlated. For example, various skin and hair conditions, treatment regimens, product usage parameters, environmental exposures and various aesthetic and medically relevant parameters.

Certain regions/functions of the cartridge are contemplated, including, an on board reagents region, a sample module, a detection module and/or a sample recovery module. One aspect of the invention is the ability to combine a series of sequential modules into a singular device, that when utilized may be inserted in to an automated instrument in such a fashion that optical signatures and patterns may be optically integrated and corresponded to, via software algorithms and database comparison, both known and presumptive diagnoses and associated recommendations.

The cartridge has the following functionalities that are modularized: an integrated on-board reagent module that is capable of storing pre-loaded fluids, powders, gels, films, encapsulated particles and the like in single or multiple separated regions, in such a fashion that upon activation the reagents are released in a controlled fashion and allowed to flow to the next module using manual pressure, gravity, wicking materials, hydrophobic/hydrophilic surface gradients, thermal expansion and/or gas driven forces.

Examples of reagents and buffer fluids used to expose skin and hair samples include: ph buffers, lysing agents, skin dissolving chemicals, enzymes, antibodies, antigens, analyte specific reagents, colorants, dyes, two-part dye compounds, nano-particles, and other functionalized materials that are sufficiently dissolvable or flowable.

A sample module, whose primary role is to accept and sufficiently isolate a variety of solid, liquid, matrix bound samples and aliquots and in so doing allow or provide sufficient reaction interface with on board reagents. This module, in one embodiment, would accept chemically functionalized adhesives that have been specifically utilized to collect, through adhesive properties, skin surface compounds, cells, exudates, naturally and artificially applied compounds, chemicals and chemical, biological and reactive and invert substances.

This sample module would be removable, replaceable and capable of being easily sealed (hermetically or otherwise) in a manner to contain the on-board reagents and intro sample, ex tape strip, so that manual shaking, agitation, heating, diffusion, mixing, dissolution, and enzymatic degrade and catalysis may occur in situ. In the case of a tape strip, the tape strip would be placed into the sample module and a reactive dye, for example, would be introduced from the reagent module, and allowed to sufficiently spread, interface, absorb, and react with skin cells and compounds present on the tape strip. These reagents would either provide direct coloration of the tape strip in the form of colorimetric, fluorescent, chemiluminescent, or otherwise optically interrogatable evidence that a reaction has occurred, or they would provide said colorimetric changes to the accompanying fluid, gel or reagent matrix.

The sample module is designed in such a manner as to be transparent in at least one region such that upon insertion of the entire cartridge into the instrument, the sample module may be continually or intermittently optically interrogated or monitored.

A secondary detection module designed in such a fashion as to allow reacted fluids and flowable products from the sample module to collect and aggregate in a region separate and distinct from the actual sample surface. This may be used for secondary sample analysis and detection and purification, amplification, separation. This detection module may be a singular well or group of wells that contain functionalized materials such as region bends, coated walls, selectively absorption matrices or optically reflective absorption properties to enhance, verify and or calibrate the optical interrogation process.

This detection module may also be fitted with optical fitters in between the detection device and the detection regions, in a manner so as to block, concentrate, control specific wavelengths of light transmitted to a reflected out of the detection region.

Additionally, this detection region may include, as part of the cartridge design, an integrated light source ranging from 200-900 that provides illumination to the individual detection regions (wells) and allows the user to directly view the associated color-changes with or without an automated reader device. This internal illumination may also be utilized as a reference calibration or control for determining sample volume, turbidity, particle size/content or may simply save as additional illumination that can be used in conjunction with the automated systems. Control of the cartridge based illumination may be provided by electrical connections that respond to “wetting” automatically as a result of fluid entering the region or may be controlled by cartridge insertion into the reader of other standard electromechanical means (switch etc.)

A final sample collection module that collects ex reagents and fluids for the purpose of providing a wicking “sink” to stimulate continuous controlled flow throughout the fluidic system and to provide a sealed, controlled recovery of fluids, compounds, DNA, RNA and other chemically and biologically relevant compounds for disposal or secondary off-cartridge analysis.

3. Pressure/Wash Vacuum Sampling

A vacuum pressure washer for obtaining biological samples from the skin, e.g., a device comprising a fluid delivery system for the purpose of obtaining biological samples from the skin. The sampler may be used with a method of applying a pressurized fluid stream, and retrieval of the fluid using a vacuum. The method of analyzing the fluid for biological samples may include an analysis method with known fluid volumes for comparing results between sample retrieval sessions. In one example, the device looks like a pen and may also include a fluid reservoir for supplying chemical reagents for enhancing the retrieval of biological samples, a secondary fluid reservoir for holding a sample stream, a transfer device for moving the analyte fluid to a microfluidic cartridge. Additional examples include a Device attachment that permits ultrasonic sonication of a skin surface, a piezoelectric device for stimulating a biochemical release from the skin and/or an optical interrogation device comprised of UV/Vis/IR light for analysis of the biological fluid.

4. Method of Care and System

A central database receives and processes information from remotes nodes, the nodes sending information collected at the physical location of the remote node (FIG. 11). The central database receives and processes the information in a comparative fashion with data contained in the central database in and sends the result back to the node. Action is then taken at the node based on the result received from the central database. The remote collection (on site) of biological sample and instant or nearly instant analysis and product recommendation provide a strong incentive for a consumer to submit to non-invasive biological sample collection with the purpose of informing the consumer and guiding them to the right product choice. This could also be used to sample other biological media such as hair, sweat, or urine, and used in other many retail settings such as gymnasiums, vitamins stores or other health and fitness outlets, (food and beverage. etc).

System. In one embodiment, a non-invasive biological sample is taken from the skin of a consumer with an adhesive tape strip at a given location, such as a store counter or salon. The tape strip by its design gives an optical contrast of the skin sample or performs or is used to perform a biochemical assay and generate an optical signal. The tape strip is then placed into a reader, the reader being optical imaging hardware connected to software programmed to perform PCA and other processing and analysis on the image collected. The image and associated PCA data, along with other data collected by GPS or associated survey information such as regional, demographic, economic, or environmental information, are then uploaded to a central database for analysis and comparison with a library of skin images and data. The central database then analyzes the incoming data and draws an association based upon a database of products to be appropriate to improve the consumer skin condition based up the skin sample analysis (image 5). The product recommendation is then sent back to the remote location where the consumer is recommended the specific product for their skin condition. A subscription fee is charged the accessor of the database (the “recommender”) and the recommendation is made for the purpose of the selling the appropriate product to the consumer. Over time and with subsequent consumer visits, the same process is used to track actual product performance and provide tangible validation or recommend another product.

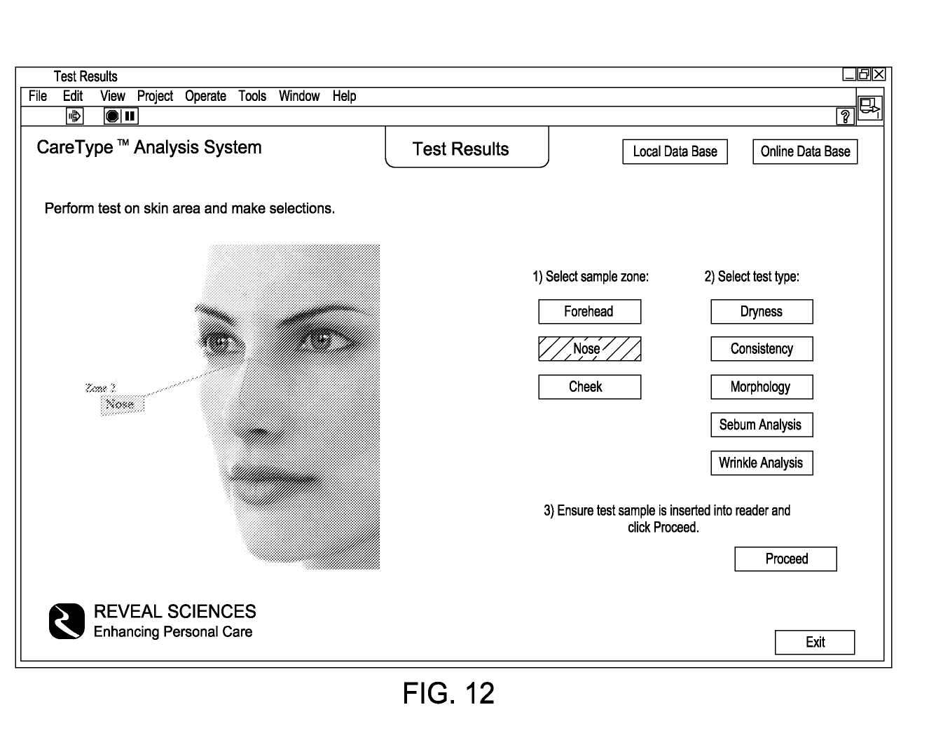

FIG. 12 shows a pre-test selection screen for use with the present invention.

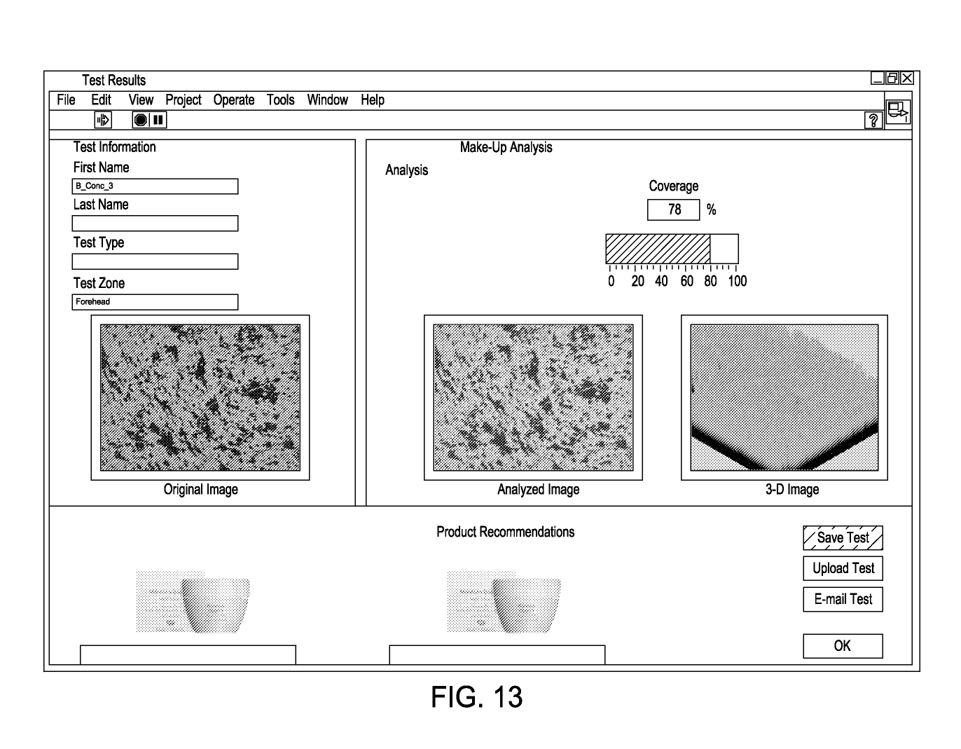

In another embodiment a tape strip is used to provide microanalysis of make-up or applied cosmetics on the skin. An adhesive strip is applied to the skin area covered with the cosmetic (FIG. 13). The strip is then taken off and placed into a reader system. The tape strip is then imaged and analyzed to observe the particulate behavior of the cosmetic of concern on the skin. Consistency and evenness of coverage, color, tone, clumping, fineness, and method of application (brush, finger, applicator device or stick) can be observed and analysis to recommend the ideal product and method of application of a cosmetic. An analysis and/or recommendation is then given to the patient.

A further embodiment, the central database is uploaded to a handheld instrument that is at the sampling location. The sample is taken and test medium is placed into the reader. The reader then performs the image and PCA analysis and database comparison on the local hard drive. The instrument then gives at an instant product recommendation.

Software: The analysis may be embodied as evaluating an image of a skin sampling tape strip based on: dryness, pore size, morphology, wrinkles, tomographic representation and/or chemical assay(s).

The present invention also includes a method of doing business comprising, a system of data collection nodes, comprising: a computational device capable of optically interrogating biological samples and obtaining user survey data; a software implemented user interface which facilitates interaction between user and computational device and serves as means for data collection, transmission, and analysis; a communication protocol to transmit data between collection node and a main server, including uploading of data to main server from collection node and downloading of data from main server to collection node; a data collection method in which data from the collection nodes is compiled, processed, and stored into a dynamically updated database on the main server, which can be searched and browsed through a web browser or software program interface. In one example, the biological samples involve living or dead tissue, such as dead skin cells or open wounds. In one example, the biological samples are test devices used to obtain a sample of tissue, such as swabs or tape strips. In another example, the biological samples are processed prior to being optically interrogated through photonic, electromagnetically radiating, chemical, biochemical, or electrical. In another example, the data collected includes images of the biological samples and survey information obtained through a questionnaire.

In another example, data is transmitted via broadband wireless or landline connection to an FTP, TCP, or e-mail server. The system may also include survey data, e.g., demographic information, product preferences, and expectations from product use, behavioral tendencies, or general preferences related to the product category or biological sample collection. The system may collect images in grayscale and/or color format and the images and survey data may be transmitted synchronously or independently of one another. In another example, data analysis occurs through one or more of the following software implemented methods: image processing algorithms which analyze images for biological, chemical, morphological, or aesthetic parameters, statistical methods to identify patterns and correlations between the data, and expert opinion or recommendation by an expert panel. The expert panel may include but not is not limited to scientists, medical professionals estheticians, marketing specialists, business developers, or any professional qualified to provide an expert opinion or recommendation based on the data. In one example, the statistical method is PCA (principle component analysis).

The system may also include one or more image processing algorithms include one or more of the following: a particle count, a LUT (look up table) filter, a particle filter, a pattern recognition, a morphological determination, a histogram, a line profile, a topographical representation, a binary conversion, or a color matching profile. The results from analysis are interpreted as product purchase recommendation, health state of the biological sample, cosmetological diagnosis, aesthetic analysis, or any plan of action based from the subsequent data analysis. The data and results from the analysis are organized and stored into the database by grouping and matching of data classified as statistically similar. The interpreted results may be viewed in one or more of the following ways: a display panel on the collection node, an e-mail message, an SMS text message, or through searching/browsing of the database in a web browser. In another aspect, the method includes a micro-analysis of applied cosmetics is performed.

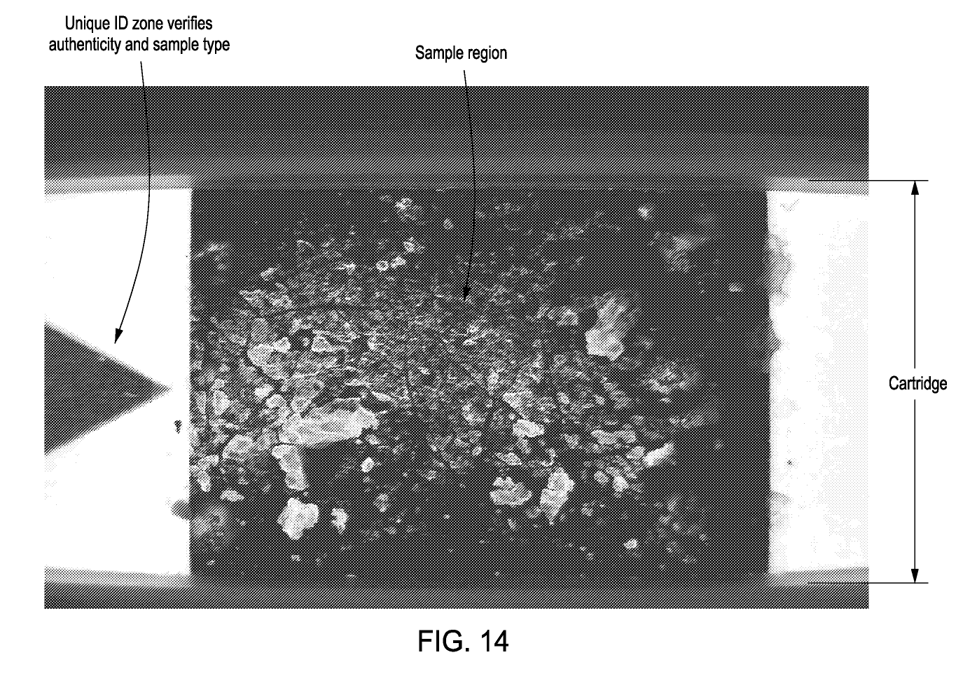

FIG. 14 shows an image of an adhesive after sampling, testing, placing in cartridge and reader system and optical interrogation and analysis by software system. Shows skin particles stained with fluorescent dye specific for certain proteins.

The present invention may also include methods for using/imaging tapes strips and chemistry on strips: A method to move/introduce fluid to test strip comprising a wicking agent embedded in adhesive. A method of sample retrieval from an adhesive surface. A method for mixing a chemical composition for indicating health markers directly on the surface of the tape strip. A method for rinsing the chemical composition from the surface. A method for collecting the rinse for analysis. A method of imaging the 3D surface of the collection surface for topographical information. A method for adding a lysing chemical composition to the surface of an adhesive tape strip after retrieving a chemical substance from a surface. An adhesive combined with micro particles a method of placing micro particles in a spatially specific location on a surface. A method of selective release of a dye resulting from the interaction of an analyte with a MIP (molecularly imprinted polymer) containing a dye, or other agents such as nonenal, acrolein, vitamin A, vitamin C.

In other embodiments, the invention includes a method for capturing chemical substances of the skin including but not limited to living cells, dead cells, and adsorbed chemical substances on the surface. A method releasing a compound from an adhesive surface for detection in the presence of a skin marker. A method of preparing disposable adhesive sample collection devices. A method of activating a chemical composition resulting from folding the surface onto itself combing the collection zone with the chemical zone. Method of making a backing material unresponsive to light. A method of using a secondary test trip to combine a sample with a reactive chemical composition. An optical identifier for the purposes of quality control and calibration and anti-counterfeiting. A method of preventing counterfeiting by embedding a substance that emits a signature wavelength detectable by an optical reader, interrogated, then processed with a calibration algorithm. A method of embedding wicking fibers into an adhesive sample collection device for allowing flow into a lateral flow membrane. A method of activating an adhesive surface resulting from the mechanical shear of removing a protective file. A method of activating an adhesive surface resulting from the exposure of the underlying surface to an activating environment, fluid or light of a specified wavelength.

A method imaging the skin by stamping or pre-treating the skin with a florescent dye or reagent composition then taking a skin sample (tape strips, swabs). A device for imaging the skin mechanically resulting in the deposition of an impression that would appear as a 3D mirror image. A device for imaging a skin sample obtained mechanically through optically interrogation with a on board camera and illuminating LEDs. A group of skin markers indicative of one or more of the following individually, or in a group: protein, oxidized protein, oil (sebum), sugars, oxidized sugars, enzymes, collagenase enzymes (MMP family, eg), ATP, Vitamins A-K, Water. A spatially oriented location designed to work with an imaging device such as a CCD or CMOS detector. An adhesive surface possessing embedded fluorescent dyes capable of absorbing broadband radiation for UV/Vis, IR near IR, far IR then emit at wavelengths specific to the properties of the dye.

Another embodiment is a tape strip device designed for integration into a microfluidic device with an onboard imaging system for the indication of skin conditions. A sample collection medium possessing an adhesive tape strip, a swab or a pressurized sample removal system combined with vacuum retrieval of said sample.

Swabs. In yet another embodiment, the present invention includes a Sample collection device possessing a chemically modified swab designed for interfacing with a microfluidic chemical reaction zone. The swab composition may be utilized by those skilled in the art including, but not limited to natural or synthetic such as cotton or polyester. A swab designed to maximize the property of fluid transfer to a microfluidic device.

Yet another embodiment is a chemically modified swab possessing a composition designed to attract the substances on the skin. The composition may be varied those skilled in the art to attract desired substances and repel interfering substances. For example, the swab may be modified to attract neutral, but polarized substances indicating the use of a polar swab such as cotton. Attract charged substances indicates the used of swabs possessing charges such as zwitterionic compositions like nitrocellulose spun fibers. The swab can attract hydrophobic substances indicating the use of a swab possessing lipophilic substances such as polyester, can be adsorbed chemical substance excreted from the skin, can be adsorbed chemical substance collected on the skin from the environment.

Yet another embodiment of the present invention includes a device for the introduction of sample for optical imaging that may include one or more of the following: a device for securing a tape strip; a device for securing a lateral flow membrane; a device for isolation of reagents; a device for holding a fluid reservoir; a device for adapting an optical filter for influencing the reading of the sample; and/or a device for microfluidic delivery of a liquid microfluidic module for selective channeling of reagents or buffers.

Yet another embodiment is a microfluidic module for collecting excess reagent or waste that may also include an adapter for securing a tape strip; an adapter for securing a sample gathering device; a sample gathering device including a cotton swab; a sample gathering device including a cotton swab equipped with a buffer or reagent pack; a sample gathering device including a cotton swab attached to a fluid delivery capsule; a n indicating device designed to combine a sample collection medium, a microfluidic chemical reaction zone with a detection zone for the imaging of the sample with or without the activating chemical composition. The sample modules may be capable of housing a 3D bed of adsorbent, a bed of adsorbent composed of x, y and z; a bed of adsorbent bed capable of separation; a separation medium that can be combined with a pressurized eluent; a separation medium capable of sequestering an analyte or dye in a zone for optical imaging; a matrix for separating solid particles from a liquid; a sample introduction zone>Reaction Zone>Imaging Zone, or a zones may be isolated, mixed or integrated depending upon reagent system.

Fluorescent dyes whose emission wavelength corresponds to an absorption wavelength for a skin marker may be used with the present invention. The present invention also includes a method of detecting a chemical marker with an indicator displacement assay; a method of isolating and enhancing the signal for a chemical marker using lateral flow membrane; a method of isolating and enhancing the signal for a chemical marker using lateral flow membrane with latex micro particles and/or gold colloids; and/or a chemical sequestration method that uses synthetic receptors, antibodies or enzymes.

The present invention also includes a method of sample retrieval from an adhesive surface, a method for mixing a chemical composition for indicating health markers directly on the surface of the tape strip; a method for rinsing the chemical composition from the surface; a method for collecting the rinse for analysis; a method of imaging the 3D surface of the collection surface for topographical information; and/or a method for adding a lysing chemical composition to the surface of an adhesive tape strip after retrieving a chemical substance from a surface.

5. Hydration Retention Profiling

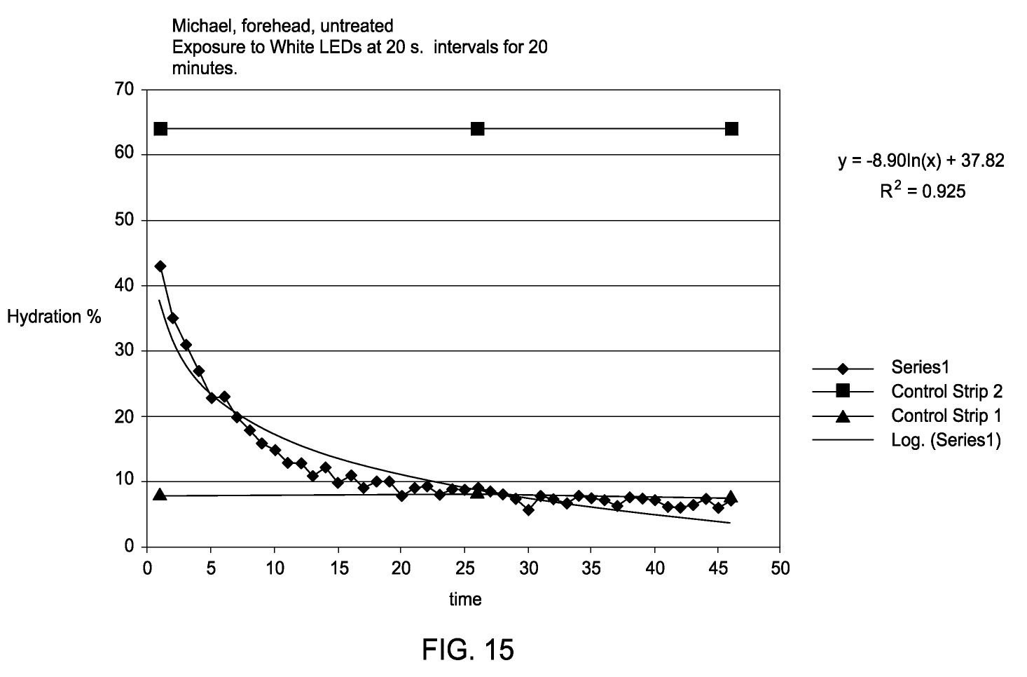

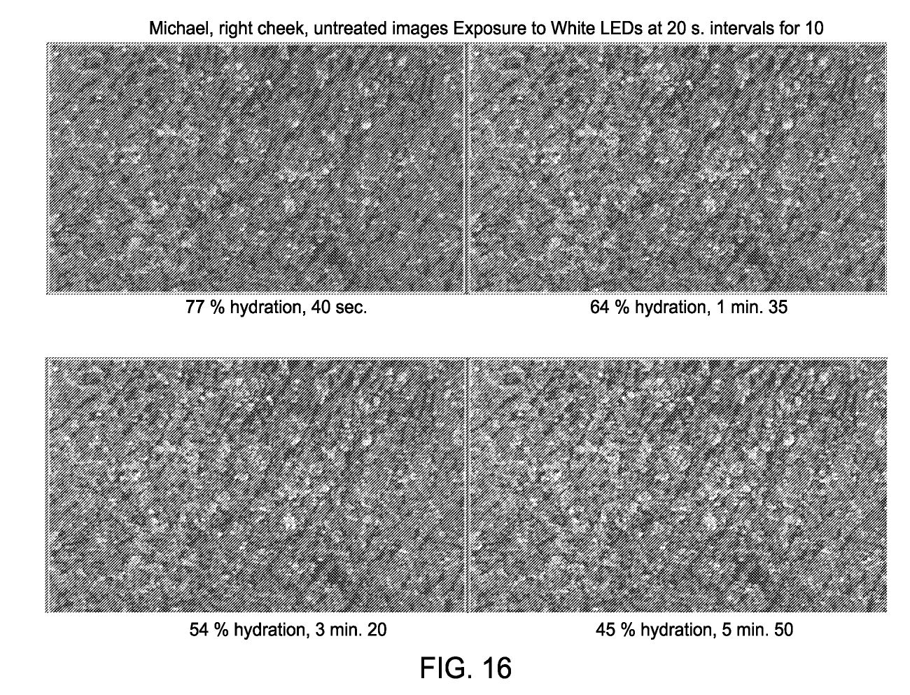

In certain embodiments an adhesive is used to collect a skin sample, and that sample is imaged over time. Analyzing logarithmic trend from the performance of the skin sampled at time intervals after tape stripping to assess how moisture is retained in the stratum corneum. The method can be used to look at the decrease in moisture levels over time, as moisture evaporates and the skin samples dries, and becomes lighter in appearance. The logarithmic curve that results from plotting the data points is show below in FIG. 15. FIG. 16 shows the images captured that are the basis for the curve in FIG. 15. The images show a clear trend of increasing whiteness in the skin flakes showing dehydration over time.

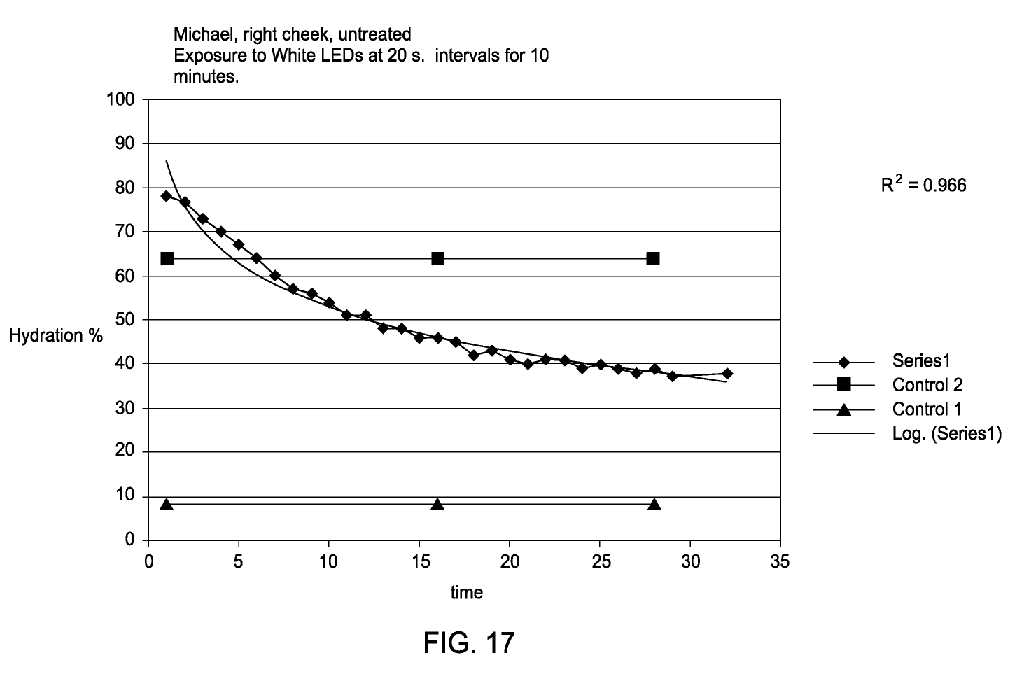

Different areas of the body, having different levels of skin hydration, will produce unique curves. FIG. 17 below shows the resultant curve from the skin sample from the cheek as compared to the forehead sample in FIG. 15.

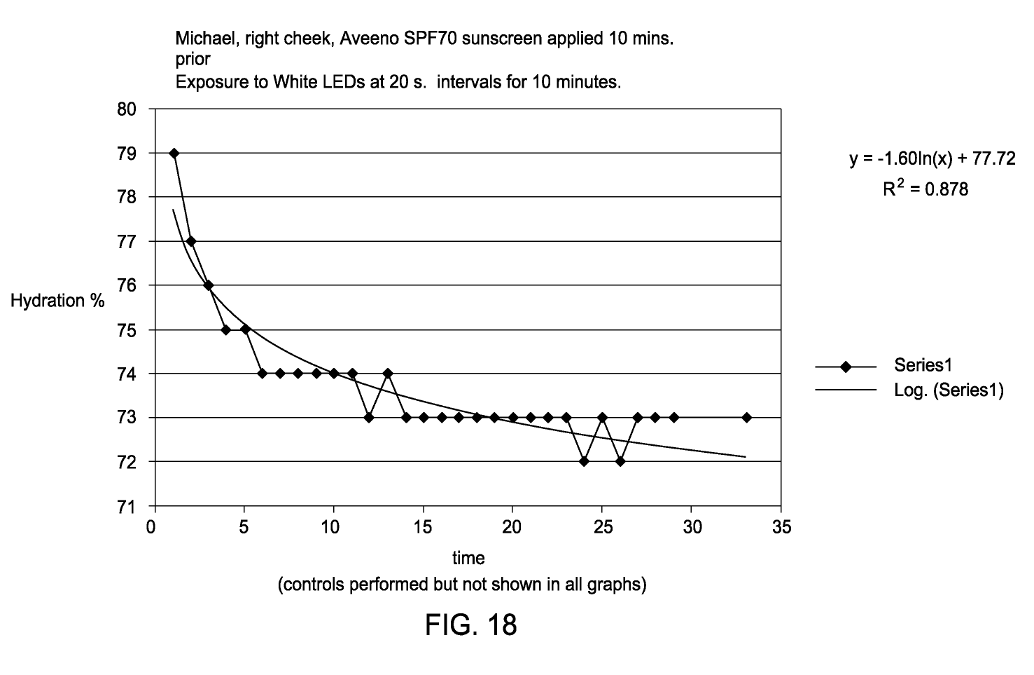

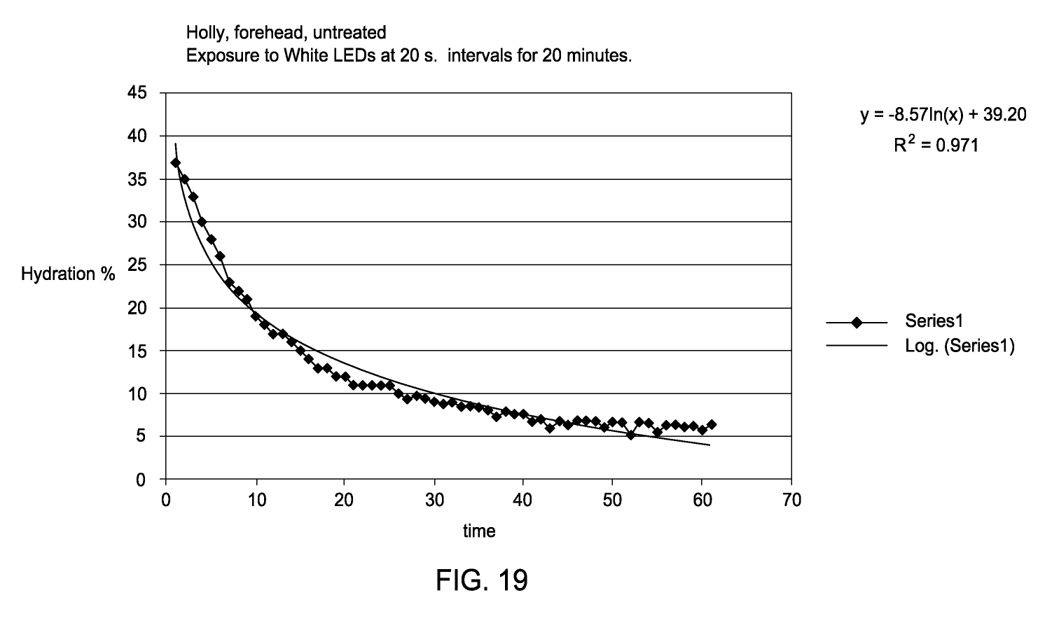

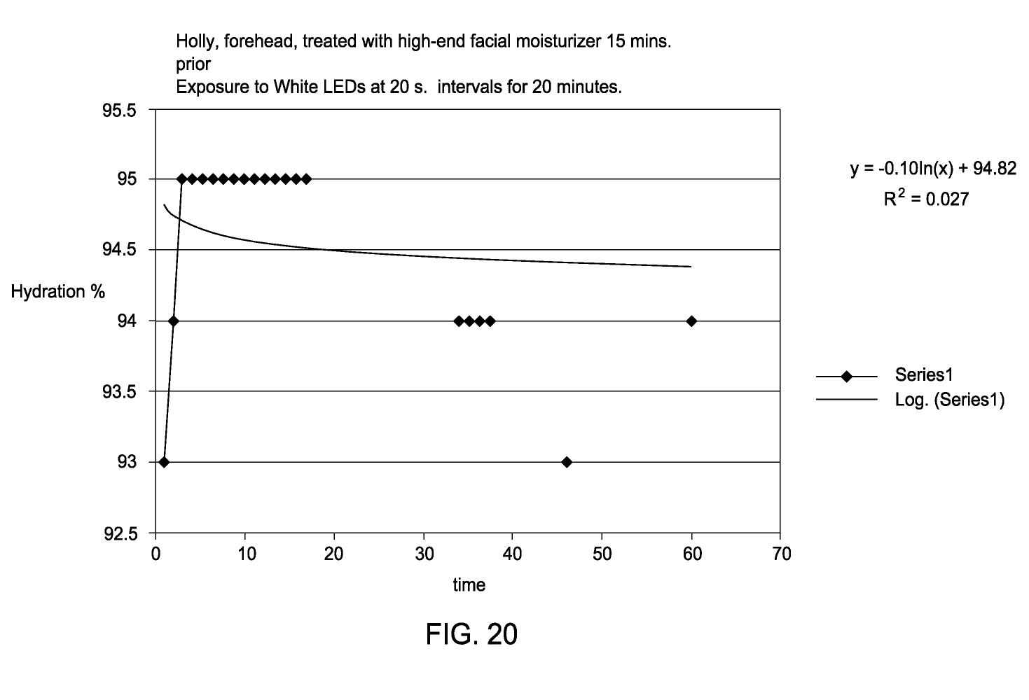

The above testing can also be used to measure the performance of a consumer product, such as a moisturizer. The same protocol above is carried out with a sample of skin previously treated with a moisturizing substance. The difference in the curve indicating the rate of dehydration of the skin then can then be used to judge the effectiveness of the moisturizer. FIG. 18 shows the application of a moisturizing sunscreen product and its effect on the curve. The curve shows the skin flakes were slower to dehydrate, and stayed ultimately at a higher level of hydration at the end of the curve. FIGS. 19 and 20 show the same principle with a moisturizer, and a different test subject.

This curve can be plotted and analyzed in different testing circumstances. In one example the imaging is done as above while the sample dehydrates. Then a hydrating factor is introduced such as an increase in humidity in the test strip environment. Subsequent images are then captured and the nature of the curve is used to characterize the rehydration performance of the sample.

Numerous factors can be introduced to the sample and used to see how they influence the performance of the skin by observing the changes in the logarithmic curve. These include processes such as heating the sample, dosing with difference wavelengths of light such as U.V. light.

Multiple adhesive samples can be taken from the same location on the skin and plotted in the same fashion as disclosed above. A basic hydration retention curve can be plotted and looked at to observe the difference in moisture retention at different depths of the skin surface.

The above testing protocols can be used together to observe how one factor influences another. For example, different products can be tested to examine their impact on hydration retention and rehydration along the logarithmic curve at different layers of the skin surface.

Products can also be looked at to observe their behavior under U.V. light. The effect of U.V. light on the skin behavior and how that is affected by U.V. light can be looked at as well. In one embodiment sunscreen efficacy is examined with this method. Sunscreen is placed on the skin and a sample is taken with the adhesive. The adhesive is then placed in the system and an image is captured under U.V. light. The amount of background fluorescence can be measured as an indicator of how much U.V. light is blocked by the sunscreen on the skin over time.

Numerous solutions, products, or compositions can be applied to analyze their affect on the skin such as vitamin.

In one embodiment the device and data are used to collect information about how a local environment, such as a work place or home, is affecting skin condition over time. A tape strip sample of an area of skin is collected and imaged at regular intervals throughout a period of time, such as a full workday, to assess how conditions are affecting the skin. For example, the rate of skin hydration from controlled ventilation, i.e., heating or air conditioning can be assessed. Subsequent data sets can be collected after the application of, for example, a moisturizing product to assess its' effectiveness and overall skin condition.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. A surface sampling device comprising:
a backing and at least two adhesive regions on the backing, wherein the adhesive in the adhesive regions is selected to capture a surface sample, wherein one or more agents are disposed in the adhesive region capable of interacting with the surface sample to aid in measuring one or more components of the sample.

2. The device of claim 1, wherein the sample comprises skin.

3. The device of claim 1, wherein the sample comprises one or more chemical species.

4. The device of claim 1, wherein the backing comprises a disposable card comprised from cardboard or vinyl sized for a cartridge.

5. The device of claim 1, wherein the adhesive is selected to remove skin cells from the stratum corneum.

6. The device of claim 1, wherein the device further comprises one or more membranes selected from nitrocellulose, UVPE, PVDF, hydrophobic membranes known to those skilled in the art of immunosorbent assays.

7. The device of claim 1, wherein the adhesive surface and the backing surface are selected to maximize the imaging capabilities of an imaging device through minimizing, maximizing or mixing reflective, absorbance and transmittance properties.

8. The device of claim 1, further comprising an optical barcode (unique id).

9. The device of claim 1, wherein the backing comprises a background with a random colored pattern for security, calibration and test validation interpretable by an algorithm processing digital signal from a camera.

10. The device of claim 1, wherein the backing comprises a thermochromic background material responsive to heat.

11. The device of claim 1, wherein the backing comprises at least one of an agent that is responsive to pressure and changes color; a translucent material that allows a simultaneous measurement of transmitted and reflected light; that is electrochromic; that changes color due to wetting, or pH or other specific chemical reaction; that comprises a phosphorescent compound that provides spontaneous illumination when expose to light of specific wavelength; comprises a chemiluminescent activated by a skin metabolite that then transmits light through regions absence of skin; and that responds to mechanical deformation by releasing embedded capsules of an activating chemical, dye or buffer.

12. A sampling device with two or more adhesive regions that have varying adhesive qualities such that upon use the sampling device gathers different amounts of sample in distinct regions.

13. The device of claim 12, wherein the adhesive surface comprises at least one of a preloaded region with an analyte specific reagent, such as a synthetic receptor; releases a dye upon experiencing a change in pressure; comprises a chemical composition for indicating health conditions; or allows flow to a subsequent surface.

14. The device of claim 12, wherein the device comprises an MIP adhesive thin film sandwiched beneath a backing material and an adhesive for removing tissue.

15. An adhesive composition for removing proteins, removing oils or capturing enzymes comprising an adhesive for removing the stratum corneum in different thicknesses for chemical testing.

16. The composition of claim 15, wherein the adhesive substance embedded with a functional chemical for enhancing the image of a collected sample.

17. The composition of claim 15, wherein the adhesive comprises an optical dye that emits in the visible range.

18. The composition of claim 15, wherein the adhesive comprises an optical dye that selectively interacts with protein, oxidized protein or lipids.

19. The composition of claim 15, wherein the adhesive comprises natural adhesives synthetic adhesives, drying adhesives, contact adhesives thermoplastic adhesives, reactive adhesives, UV and light curing adhesives or pressure sensitive adhesives.

20. The composition of claim 15, wherein the adhesive comprises chemical and physical properties that detector one or more chemical species in the skin, is optimized for optical imaging, illuminated from LED's in the IR, visible and UV wavelengths.

21. The composition of claim 15, further comprising one or more indicating dyes, buffers and activators for indicating the presence of skin markers.

22. The composition of claim 15, wherein the adhesive comprises dyes, buffers and activators for highlighting a group of skin properties such as moisture, dryness, irritation, wrinkles or sun damage.

23. The composition of claim 15, wherein the adhesive comprises two or more adhesives selected are porous, hydrophilic, hydrophobic, double-sided and hydrogels.

24. The composition of claim 15, wherein the adhesive captures topographical information from the surface to which it is attached.

25. The composition of claim 15, wherein the adhesive comprises one or more agents that simultaneously monitors pH, moisture content, and oil.

26. The composition of claim 15, further comprising an agent that disrupts cell membranes.

27. The composition of claim 15, further comprising one or more reagents selected from: a buffer, a dye, an activator a synthetic receptor or linker.

28. An assay comprising and adhesive for sample collection that comprises a receptor and a dye whose optical properties change in the presence of oxidized protein.

29. A module comprising an adhesive for obtaining a sample device, comprising:
a tape strip collected sample washed from the surface, the collected in a sample chamber;
a sample chamber that could be imaged directly;
a sample chamber whose exposure to an eluent results in the flow of the chemical marker to an imaging zone using microfluidics;
a microfluidic system comprising a lateral flow membrane; and
a lateral flow membrane characterized as hydrophilic, hydrophobic or super hydrophobic.

30. A method of assessing skin condition comprising:
collecting a skin sample using an adhesive strip that collects a sample of skin;
measuring the intensity level of one or more analytes that interact with the skin sample; and
analyzing the contrast levels against the background by imaging the strip comprising the skin sample over time.

31. The method of claim 30, wherein the adhesive strip comprises a backing and at least two adhesive regions on the backing, wherein the adhesive is selected to capture a surface sample, wherein one or more agents are disposed in the adhesive region capable of interacting with the surface sample to aid in measuring one or more components of the sample.

32. The method of claim 30, wherein the sample comprises one or more chemical species that interact with the one or more agents.

33. The method of claim 30, wherein the backing comprises a disposable card comprised of cardboard or vinyl and the backing is sized for a cartridge having predetermined dimensions.

34. The method of claim 30, wherein the adhesive is selected to remove skin cells from the stratum corneum.

35. The method of claim 30, wherein the performance of a skin product is assessed by sampling skin with an adhesive strip used to collect a sample of skin treated with a product and analyzing the contrast levels against the background by imaging the sample strip over time.

36. The method of claim 30, wherein the effect of a local environment on skin condition comprising an adhesive strip used to collect a sample of skin and analyzing the contrast levels against the background by imaging the sample strip over time and comparing to a control skin sample.

37. An adhesive strip comprising:
an adhesive region for sampling a skin sample, a pressure sensitive dye to assess pressure, and a moisture sensitive pad to assess moisture on the skin.
